(19) United States
(12) Patent Application Publication
(10) Pub. No.: US 2020/0306757 A1
(43) Pub. Date: Oct. 1, 2020

(54) QUANTUM PLASMONIC RESONANT ENERGY TRANSFER AND ULTRAFAST PHOTONIC PCR

(71) Applicant: NATIONAL UNIVERSITY OF SINGAPORE, SINGAPORE (SG)

(72) Inventor: Luke P. LEE, Orinda, CA (US)

(21) Appl. No.: 16/760,983

(22) PCT Filed: Oct. 31, 2018
(86) PCT No.: PCT/IB2018/001363

§ 371 Date: May 1, 2020

Related U.S. Application Data
(60) Provisional application No. 62/580,372, filed on Nov. 1, 2017.

(51) Int. Cl. B01L 3/00; B01L 7/00; C12Q 1/6806; G01N 21/65

(52) U.S. Cl. CPC B01L 3/502761; B01L 7/52; B01L 3/50273; C12Q 1/6806; G01N 21/65; B01L 2200/0668; C12Q 2565/628; B01L 2300/0825; B01L 2300/1861; B01L 2400/0406; B01L 2400/086; C12Q 2531/113; B01L 2300/0654

(57) ABSTRACT

A rapid and precision molecular diagnostic chip making use of quantum plasmonic resonance energy transfer is disclosed for performing ultrafast polymerase chain reaction (PCR). The chip includes functionally graded microfluidic structures capable of receiving and conveying a sample using self-powered capillary pumping and capable of performing on-chip separation and target pathogen lysis. The chip can include optical traps to selectively trap and enrich various constituents of the sample, such as cell-free deoxyribonucleic acids (cfDNAs) and exosomes. In some cases, a processing device can receive a diagnostic chip, induce PCR within the diagnostic chip, and optionally detect diagnostic data from the samples within the diagnostic chip.

### CROSS REFERENCE TO RELATED APPLICATIONS

The present application claims the benefit of U.S. Provisional Application No. 62/580,372 filed on Nov. 1, 2017 and entitled “QUANTUM PLASMONIC RESONANT ENERGY TRANSFER AND ULTRAFAST PHOTONIC PCR,” which is hereby incorporated by reference in its entirety.

### TECHNICAL FIELD

The present disclosure relates to medical or scientific diagnostic equipment generally and more specifically to polymerase chain reaction equipment.

### BACKGROUND

Polymerase Chain Reaction (PCR) is a fundamental tool with applications in many industries, such as healthcare and medicine, veterinary practice, agriculture, food, and forensics, among others. PCR enables the amplification of deoxyribonucleic acid (DNA), which provides a basis for the detection and analysis of DNA, such as through fluorescent markers. Thus, PCR is an important tool for many areas of research (e.g., invention of new drugs in pharmacogenomics) and diagnostics (e.g., diagnosing a patient for personalized healthcare or tracking the spread of pathogens in epidemiology). Generally, PCR often relies on repeated thermal cycling to melt or denature the DNA and then replicate the DNA through the use of a DNA polymerase, such as Taq polymerase.

Current PCR techniques involve cumbersome and labor-intensive sample preparation steps, such as DNA extraction, purification, and quantification, which may take hours to complete (e.g., 1-3 hours in some cases). Further, commercial PCR devices use large heating elements with high power consumption, such as desktop systems requiring 300-600 Watts of alternating current to function.

Current PCR techniques use thermal cycling equipment to control the temperature of the DNA sample. Often, sample tubes, sample wells, or other chambers contain the DNA samples (e.g., blood or other materials, sometimes with a carrier fluid) in amounts on the order of tens or hundreds of microliters or more during the thermal cycling. Thermal cycling equipment is then used to apply heat and/or cooling to the sample tubes, sample wells, or other chambers, which then inducing heating or cooling of the DNA sample by conducting heat through the walls of the sample tube, sample well, or other chamber. The equipment associated with current PCR techniques can be expensive, large, heavy, and can consume substantial power during operation. The sample vessels can be relatively large, on the order of tens of microliters. Further, the entire processing time to amplify the DNA is often approximately 50-60 minutes or more. Thin film heaters may be used to try and control temperature of static microfluidic-based PCR systems, however such heaters require a complicated fabrication process to integrate the thin film heater and resistance temperature detection sensor on the chip. Further, current microfluidic-based PCR systems still rely on standard sample extraction, isolation, and preparation techniques, which can be cumbersome, time consuming, and costly.

Prior to performing PCR on a sample using current techniques, it may be necessary to prepare the sample. Various sample preparation techniques may require machinery and equipment, such as centrifuges, and numerous steps and tedious procedures. For example, a blood sample from a patient may need to undergo numerous cycles on a centrifuge between various collection, lysing, washing, and elution steps. In some cases, other sample preparation techniques may be used.

Current PCR techniques may also require the use of numerous consumables, such as sample chambers, transfer chambers and equipment (e.g., micropipette tips or swabbing materials), and other multi-use or single-use consumables, which can result in high costs per test.

### SUMMARY

The term embodiment and like terms are intended to refer broadly to all of the subject matter of this disclosure and the claims below. Statements containing these terms should be understood not to limit the subject matter described herein or to limit the meaning or scope of the claims below. Embodiments of the present disclosure covered herein are defined by the claims below, not this summary. This summary is a high-level overview of various aspects of the disclosure and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this disclosure, any or all drawings and each claim.

Certain aspects of the present disclosure include an ultrafast diagnostic device, comprising: a sample input for accepting a sample containing desired particles; a fluid network comprising a plurality of fluid pathways extending distally away from the sample input, wherein the fluid network comprises: a separation zone comprising one or more cavities configured to retain undesired particles from the sample, wherein the one or more cavities are coupled to the plurality of fluid pathways to permit passage of the desired particles through the separation zone; a reaction zone comprising a plurality of plasmonic nanocavities fluidly coupled to the plurality of fluid pathways, wherein each plasmonic nanocavity comprises opposing walls each comprising a layer of plasmonic material, wherein the opposing walls of the plasmonic nanocavity are spaced apart by a distance of approximately 5 nanometers or less; and a window permitting transmission of light into and out of the plurality of plasmonic nanocavities of the reaction zone, wherein the window permits transmission of light having wavelengths in the visible spectrum, the infrared spectrum, or the ultraviolet spectrum.

In some cases, the opposing walls of the plasmonic nanocavities are spaced apart by a distance at or less than 3 nm. In some cases, the fluid network further comprises: a pumping zone comprising one or more capillaries sized to induce motive force in the sample through capillary action upon introduction of the sample into the sample input. In some cases, the one or more cavities of the separation zone form a functional gradient having openings sized to accept the undesired particles. In some cases, each of the one or more cavities of the separation zone extend from the one of the plurality of fluid pathways within the separation zone to permit gravitational settling of the undesired particles within the cavity. In some cases, the fluid network further comprises: a lysing zone comprising one or more cavities for receiving lysable particles of the sample and a set of electrodes positioned to supply an electrical current at the one or more cavities to facilitate lysing the lysable particles, wherein the desired particles of the sample are located within the lysable particles. In some cases, the set of external electrical contacts are couplable to an external device for supplying the electrical current to the set of electrodes. In some cases, the one or more cavities of the separation zone are sized to accept blood cells. In some cases, each plasmonic nanocavity of the reaction zone is sized to accept a single double helix of nucleic acid. In some cases, the opposing walls of each plasmonic nanocavity of the reaction zone further comprises a layer of dielectric material. In some cases, each plasmonic nanocavity of the reaction zone further comprises a polymerase reagent. In some cases, the polymerase reagent is a lyophilized polymerase reagent.

Certain aspects of the present disclosure include a diagnostic system comprising a diagnostic chip comprising any of the ultrafast diagnostic devices as described above and a processing device for processing the diagnostic chip, wherein the processing device comprises: a receptacle sized to accept the diagnostic chip; a light source positioned to illuminate the reaction zone when the diagnostic chip is positioned within the receptacle; and a processor coupled to the light source to control application of light to the reaction zone to induce plasmonic resonance in the plasmonic nanocavities of the reaction zone. In some cases, the processing device further comprises a detector coupled to the processor and positioned to detect electromagnetic emissions from the reaction zone of the diagnostic chip.

Certain aspects of the present disclosure include a method of preparing materials, comprising: receiving a sample containing desired particles at a sample input of a diagnostic device; conveying the desired particles through a fluid network in a distal direction, wherein conveying the desired particles through the fluid network comprises: conveying the sample into a separation zone, wherein conveying the sample into the separation zone comprises separating undesired particles from the sample and conveying the desired particles through the separation zone; and conveying the desired particles into plasmonic nanocavities of a reaction zone, wherein each plasmonic nanocavity comprises opposing walls each comprising a layer of plasmonic material, wherein the opposing walls of each plasmonic nanocavity are spaced apart by a distance of approximately 5 nanometers or less; and transmitting light into each of the plasmonic nanocavities through a window, wherein the light is selected from the group consisting of infrared light, visible light, and ultraviolet light.

In some cases, conveying the desired particles into plasmonic nanocavities of the reaction zone further comprises conveying each of the desired particles to a unique one of the plasmonic nanocavities. In some cases, conveying each of the desired particles to unique ones of the plasmonic nanocavities comprises conveying double helixes of nucleic acids to unique ones of the plasmonic nanocavities. In some cases, conveying the desired particles through the fluid network further comprises pumping the desired particles through the fluid network using capillary action. In some cases, conveying the sample into the separation zone further comprises conveying the sample through a functional gradient having openings sized to accept the undesired particles, wherein separating the undesired particles from the sample comprises trapping the undesired particles in the functional gradient. In some cases, trapping the undesired particles in the functional gradient includes permitting the undesired particles to gravitationally settle into one or more cavities of the separation zone. In some cases, lysing lysable particles occurs within a lysing zone of the fluid network located distally from the separation zone. In some cases, lysing the lysable particles comprises applying an electrical current to the separation zone. In some cases, separating undesired particles from the sample comprises separating blood cells from a blood sample. In some cases, the opposing walls of each plasmonic nanocavity of the reaction zone further comprises a layer of dielectric material.

Certain aspects of the present disclosure include a diagnostic system, comprising: a diagnostic chip comprising a sample input for accepting a sample containing desired particles and a fluid network, the fluid network comprising: a separation zone comprising one or more cavities configured to retain undesired particles from the sample, wherein the one or more cavities are coupled to a plurality of fluid pathways of the fluid network to permit passage of the desired particles through the separation zone; and a reaction zone comprising a plurality of plasmonic nanocavities fluidly coupled to the plurality of fluid pathways, wherein each plasmonic nanocavity comprises opposing walls each comprising a layer of plasmonic material, wherein the opposing walls of the plasmonic nanocavity are spaced apart by a distance of approximately 5 nanometers or less; and a processing device for processing the diagnostic chip, wherein the processing device comprises: a receptacle sized to accept the diagnostic chip; a light source positioned to illuminate the reaction zone when the diagnostic chip is positioned within the receptacle; and a processor coupled to the light source to control application of light to the reaction zone to induce plasmonic resonance in the plasmonic nanocavities of the reaction zone.

In some cases, the processing device further comprises a detector coupled to the processor and positioned to detect electromagnetic emissions from the reaction zone of the diagnostic chip.

Certain aspects of the present disclosure include a diagnostic method, comprising: preparing materials according to the method of any of examples 13-22; and inducing plasmonic resonance in the plasmonic nanocavities, wherein inducing plasmonic resonance comprises illuminating the reaction zone with light.

In some cases, heating the desired particles comprises inducing the plasmonic resonance, and wherein cooling the desired particles comprise ceasing illuminating the reaction zone with light. In some cases, illuminating the reaction zone with light includes using a light source, and wherein detecting electromagnetic emissions comprises illuminating the reaction zone using the light source to evoke the electromagnetic emissions. In some cases, the method further comprises storing the electromagnetic emissions as image data; and analyzing the image data to determine a diagnostic inference. In some cases, analyzing the image data comprises using a deep neural network to determine the diagnostic inference. In some cases, analyzing the image data comprises: transmitting the image data using a network interface, wherein transmitting the image data using the network interface results in the image data being applied to a deep neural network to generate the diagnostic inference when the transmitted image data is received; and receiving the diagnostic inference using the network interface.

Certain aspects of the present disclosure include a method of preparing a chip, comprising: providing a substrate having a plurality of walls defining a plurality of passages, wherein the plurality of passages includes one or more passages having a width of at or less than 100 nm; oxidizing surfaces of the plurality of walls to form an oxidization layer; depositing a plasmonic material on the oxidization layer; and loading reagent into the plurality of passages.

In some cases, providing the substrate comprises providing a silicon substrate, and wherein oxidizing the surfaces of the plurality of walls forms a layer of silicon dioxide. In some cases, the plurality of passages includes one or more passages having a width of at or less than 40 nm. In some cases, the plurality of passages includes one or more passages having a width of at or less than 10 nm. In some cases, depositing the plasmonic material comprises depositing gold. In some cases, loading reagent comprises loading lyophilized reagent into the plurality of passages. In some cases, loading lyophilized reagent comprises loading lyophilized polymerase chain reaction reagents. In some cases, loading reagent comprises: loading a first reagent into a first set of the plurality of passages; and loading a second reagent into a second set of the plurality of passages. In some cases, the method further comprises loading nucleic acid probes into the plurality of passages. In some cases, loading nucleic acid probes comprises: loading a first nucleic acid probe into a first set of the plurality of passages; and loading a second nucleic acid probe into a second set of the plurality of passages. In some cases, each of the plurality of passages have an open top, and wherein the method further comprises sealing the open top of each of the plurality of passages. In some cases, sealing the open top of each of the plurality of passages comprises sealing each of the plurality of passages with a window permitting transmission of light into and out of the passage.

Certain aspects of the present disclosure include a method for imaging electron transfer, comprising: positioning a plasmonic nanoantenna adjacent target tissue; irradiating the plasmonic nanoantenna with electromagnetic energy to induce the plasmonic nanoantenna to emit emitted electromagnetic energy, wherein the emitted electromagnetic energy is associated with electron transfer of the target tissue; measuring emitted electromagnetic energy from the plasmonic nanoantenna.

In some cases, the target tissue is an ion channel of a membrane. In some cases, the ion channel is a cytocrome c protein of a mitochondrial membrane. In some cases, irradiating the plasmonic nanoantenna with electromagnetic energy comprises irradiating the plasmonic nanoantenna with light.

Certain aspects of the present disclosure include a method for biological intervention, comprising: positioning a plasmonic nanoantenna adjacent target tissue; and manipulating electron transfer of the target tissue by irradiating the plasmonic nanoantenna with electromagnetic energy.

In some cases, the target tissue is an ion channel of a membrane. In some cases, the ion channel is a cytocrome c protein of a mitochondrial membrane. In some cases, irradiating the plasmonic nanoantenna with electromagnetic energy comprises irradiating the plasmonic nanoantenna with light.

### BRIEF DESCRIPTION OF THE DRAWINGS

The specification makes reference to the following appended figures, in which use of like reference numerals in different figures is intended to illustrate like or analogous components.

FIG. 1 is a schematic diagram of a plasmonic PCR system according to certain aspects of the present disclosure.

FIG. 2 is a top view of a diagnostic chip according to certain aspects of the present disclosure.

FIG. 3 is a side cross sectional view of a diagnostic chip according to certain aspects of the present disclosure.

FIG. 4 is a front cross-sectional view of a lysing zone of a diagnostic chip according to certain aspects of the present disclosure.

FIG. 5 is a schematic side view of an ultrafast diagnostic device according to certain aspects of the present disclosure.

FIG. 6 is a flowchart depicting a process for conducting on-chip filtering, lysing, and reacting according to certain aspects of the present disclosure.

FIG. 7 is a combination axonometric diagram of a set of pillars of a diagnostic chip and a schematic cross-sectional diagram depicting the passageway between the pillars according to certain aspects of the present disclosure.

FIG. 8 is a schematic cross-sectional diagram depicting plasmon-assisted denaturing of a nucleic acid within a plasmonic nanocavity according to certain aspects of the present disclosure.

FIG. 9 is a schematic cross-sectional diagram depicting plasmon-assisted elongation of a nucleic acid within a plasmonic nanocavity according to certain aspects of the present disclosure.

FIG. 10 is a schematic cross-sectional diagram depicting plasmon-assisted trapping of an exosome within a plasmonic nanocavity according to certain aspects of the present disclosure.

FIG. 11 is a schematic diagram depicting a multiplexed reagent-loaded reaction zone according to certain aspects of the present disclosure.

FIG. 12 is set of schematic top view diagrams depicting a diagnostic chip with a multiplex reagent-loaded reagent zone according to certain aspects of the present disclosure.

FIG. 13 is a schematic diagram depicting a processing device for processing diagnostic chips according to certain aspects of the present disclosure.

FIG. 14 is a schematic diagram depicting plasmonic heating and cooling according to certain aspects of the present disclosure.

FIG. 15 is a flowchart depicting a process for collecting and analyzing a sample according to certain aspects of the present disclosure.

FIG. 16 is a top view of a diagnostic chip with thermal lysing according to certain aspects of the present disclosure.

FIG. 17 is a flowchart depicting a process for preparing a diagnostic chip according to certain aspects of the present disclosure.

FIG. 18 is a schematic diagram depicting a lysis zone of a diagnostic chip according to certain aspects of the present disclosure.

FIG. 19 is a side view of a nanocrescent antenna according to certain aspects of the present disclosure.

FIG. 20 is a side cutaway view of a multilayer nanocrescent antenna according to certain aspects of the present disclosure.

FIG. 21 is a schematic side view of a nanoantenna usable to effect an ion channel of a membrane according to certain aspects of the present disclosure.

### DETAILED DESCRIPTION

Certain aspects and features of the present disclosure relate to leveraging quantum electron transfer for biological applications, such as performing polymerase chain reactions. As used herein, quantum electron transfer can refer to quantum plasmon energy transfer and quantum biological electron transfer (QBET). QBET can refer to the coupling of transferring electrons with quantum mechanical tunneling in biological systems.

Certain aspects and features of the present disclosure relate to a diagnostic chip capable of performing ultrafast polymerase chain reaction (PCR) by taking advantage of quantum plasmon resonance energy transfer. The chip can include functionally graded microfluidic structures capable of receiving and conveying a sample using self-powered capillary pumping and capable of performing on-chip separation and target pathogen lysis. The chip can include optical traps to selectively trap and enrich various constituents of the sample, such as cell-free deoxyribonucleic acids (e.g., codas) and exosomes. In some cases, a processing device can receive a diagnostic chip, induce PCR within the diagnostic chip, and optionally detect diagnostic data from the samples within the diagnostic chip.

In some cases, the diagnostic chip can include an array of pillars or other structures that define passages (e.g., pathways) and gaps therethrough. The array of pillars can extend from, be situated on, be coupled to, or be otherwise integrated with a substrate. The substrate can be formed of any suitable material, such as Poly (methyl methyacrylate), which may also be used as a base material for pillars or other features of the diagnostic chip. The chip can be fabricated in any suitable fashion, including through photo resistive etching, molding with Polydimethylsiloxane, laser machining, or laser embossing. The entire chip or portions of the chip may be transparent or translucent to light, such as infrared, visible, and/or ultraviolet light. In some cases, a portion of the chip that is transparent or translucent to light can be a window.

The pillars in the array of pillars can be any suitable shape, although in some cases the pillars can be hexagonal in shape (e.g., cross section) and arranged in a hexagonal array. The hexagonal shape can provide a large surface area. The array of hexagonal pillars can promote hot spot coupling and have other advantages as disclosed herein. Other shapes can be used, such as circular, triangular, bowtie, crescent, and others. The passages defined at least in part by the pillars can make up a fluid network capable of conveying fluid through the diagnostic chip. The fluid network can convey fluid, such as a sample, from a sample input through different parts of the chip. As used herein, the term “through” with reference to conveying samples, fluids, or particles with respect to the fluid network or any parts of the fluid network can include transporting the samples, fluids, or particles into and/or along the parts of the fluid network, but not necessarily out of the fluid network or any parts of the fluid network. Therefore, conveying a desired particle through the fluid network can include conveying the desired particle into the fluid network, along one or more passages, and into a reaction location (e.g., reaction well), without ever exiting the fluid network.

In some cases, the array of pillars can form a nanofluidic gradient generator due to gradients in the heights of the passages and/or pillars, as well as the gradient in gap between pillars (i.e. gap junctions). For example, the height for the passages and/or height of the pillars can change (e.g., become smaller) along the downstream direction within the chip. Thus, the passageway may begin tall and slowly become shorter. As well, the passages can include cavities (e.g., trenches) extending therefrom in which cell components and debris may be deposited or trapped as the sample flows through the passages. These cavities or trenches can be deeper near the sample input and become shallower as they are positioned further away from the sample input. The topology of the pillars within the chip or topology of passages or trenches within the fluid network can thus permit gravity-assisted separation of desired particles from a sample without the need for centrifuging. This type of functionally graded microfluidics can enable superior miniaturization and other improvements.

Different zones of the chip can perform various functions, such as separation, pumping, lysing, and reacting (e.g., PCR). Zones can contain portions of the fluid network, including passages or portions of passages. Different zones may be distinct from one another or may overlap one another. For example, in some cases a particular portion of the fluid network may be considered to be part of only a single zone, although in other cases the particular portion of the fluid network may be considered to be part of two or more zones. For example, features (e.g., passages) of a pumping zone may also be used to separate the sample, and may thus be also considered part of a separation zone.

In some cases, zones can be located sequentially with respect to one another. For example, a separation zone may be located upstream of (e.g., proximal to) a pumping zone, which may be located upstream of (e.g. proximal to) a lysis zone, which may be located upstream of (e.g., proximal to) a reaction zone. In some cases, fewer or more zones may be used, and in any suitable combination.

A sample input zone can receive the sample. The sample can be a fluid sample, such as blood, saliva, or exhaled condensate. In some cases, non-fluid samples (e.g., skin surface materials) can be combined with fluid before or during deposit into the sample input zone. For example, in the case of a skin swab, the surface of the skin may be swabbed and the swab may be placed in a tube containing a fluid which can facilitate flow through the fluid network after the sample is provided to the diagnostic chip. In some examples, however, a swab contains materials from the surface of the skin may be placed directly into the sample input zone and be mixed with fluid already present in or simultaneously supplied to the sample input zone to entrain the skin surface materials in the fluid. In some cases, the sample input zone can include a reservoir of carrier fluid for accepting non-fluid samples and conveying the samples through the fluid network.

In some cases, a blood sample can be used, which can be collected from a heel prick, a finger stick, a venipuncture, or otherwise. In some cases, the sample input zone can include a built-in blood draw device, such as a lancet, to initiate blood draw (e.g., via finger stick) directly into the sample input zone. In some cases, the sample input zone can be shaped to easily receive a droplet, thus facilitating manual depositing of a fluid sample. In some cases, the sample input zone can be shaped to interconnect with and/or interlock with a blood container (e.g., filled blood draw tubes) to facilitate depositing of the sample into the sample input zone. In some cases, a sample input zone can include a removable and/or replaceable cover to maintain the integrity of the fluid network from contamination. In some cases, the sample input zone can further include a filter, such as a filter designed to filter out coarse contaminants, such as dirt, from the sample before the sample proceeds down the fluid network.

A sample can include particles within a fluid. Particles can include cells, cellular structures, nucleic acids, bacteria, viruses, exosomes, vesicles, or any other non-fluid portion of a sample. In some cases, particles can include lysable particles, which can be any particle having a membrane or similar structure capable of being lysed, such as cells and exosomes. Generally, a lysable particle can include a payload coupled to or contained within the membrane of the lysable particle. Such a payload may itself be another particle. As used herein, the terms desired particle or reaction particle can refer to those particles desired to be delivered to a reaction zone for performing a particular reaction, such as PCR. For example, desired particles or reaction particles may include nucleic acids, such as DNA, including cell-free DNA. In some cases, the term desired particle can refer to a particle desired to be delivered to a subsequent zone for subsequent processing.

A separation zone can include portions of the chip (e.g., portions of the fluid network) capable of separating desired particles from a sample. The separation zone can include functionally graded microfluidics, such as described above, to separate undesired particles from desired particles. Undesired particles can remain trapped in the fluid network, such as trapped within trenches of the fluid network, while desired particles can be transported to a subsequent zone or subsequent are of the separation zone. In some examples, such as when the sample includes blood, the separation zone can separate exosomes from red blood cells, in which case red blood cells can be retained in trenches of the fluid network and the exosomes can be delivered to a subsequent zone.

A pumping zone can include portions of the chip (e.g., portions of the fluid network) capable of facilitating movement of the sample and/or particles through the fluid network. The pumping zone can include passages, portions of passages, or other features that invoke a capillary action, resulting in movement of the sample and/or particles through the fluid network. The pumping zone can partially or fully overlap the separation zone (e.g., be incorporated in the separation zone, such as pumping elements of the separation zone), although that need not be the case. The wicking capability (e.g., flow rate) of the pumping zone can be tuned by the geometry and topology of the fluid network (e.g., the geometry and topology of the pillars or other structures defining the fluid network). For example, the length and gap between hexagonal pillars can be altered to achieve a desired flow rate. The wicking capacity (e.g., volume) can be tuned by scaling the device, as well as through altering the geometry and topology of the fluid network.

A lysing zone can include portions of the chip (e.g., portions of the fluid network) capable of lysing lysable particles (e.g., exosomes) to release further particles (e.g., nucleic acids) for analysis. In some cases, lysing can be achieved by local hydroxide (2H₂O→H₃0⁺+OH⁻) generation to extract nucleic acids from the lysable particles. In some cases, local hydroxide generation can occur through the application of electrical current within the lysing zone. The lysing zone can include passages, cavities, and/or trenches. The electrical current can be generated in, through, at, and/or near (e.g., through a region in close fluid communication with) these passages, cavities, and/or trenches to generate sufficient hydroxide to lyse lysable particles located within the passages, cavities, and/or trenches. In some cases, a lysing zone can be prepared to include a reagent suitable to lyse of facilitate lysing of lysable particles, although that need not be the case.

A reaction zone can include portions of the chip (e.g., portions of the fluid network) capable of performing desired reactions, such as PCR. The reaction zone can include passages, cavities (e.g., wells), trenches or other features of the fluid network that can trap or contain reaction particles (e.g., DNA). The passages, cavities, trenches, or other features of the fluid network of the reaction zone can be prepared or pre-populated (e.g., pre-loaded) with primers, probes, and/or reagents, such as polymerase (e.g. a polymerase suitable for PCR). In some cases, a thermostable polymerase can be used. In some cases, the PCR reagent (e.g., polymerase) can be an inhibitor-resistant reagent (e.g., resistant to PCR inhibitors, such as cell-free hemoglobin). A single chip can contain any number of discrete reaction locations (e.g., passages, cavities, trenches, or other features of the fluid network where reactions are to occur).

In some cases, reagents can be lyophilized (e.g., freeze-dried) prior to being pre-loaded into a passage, cavity, or other feature of the fluid network. In some cases, lyophilization can include the use of lyoprotectants, such as trehalose, sorbitol, and glycerol, although others can be used. The use of lyophilized reagents (e.g., lyophilized polymerases) can improve the shelf life of chips and can permit chips to be stored without the need for refrigeration. Other materials that are pre-loaded into the fluid network can likewise be lyophilized.

In some cases, a multiplexed analysis can be performed by pre-loading the passages, cavities, trenches, or other features of the fluid network of different regions of the reaction zone with different primers, probes, and/or reagents. Thus, each of the different regions (e.g., multiplex regions) can provide unique analysis based on the same collection of particles from the same sample. For example, a chip can be pre-loaded with a first primer specific to a first pathogen in a first region and pre-loaded with a second primer specific to a second pathogen in a second region. Thus, when a sample is supplied to the sample input zone and reaction particles flow into the reaction zone, some reaction particles will flow into the first region and some reaction particles will flow into the second region. When light is applied to the reaction zone to perform a reaction (e.g., PCR) and/or analysis, two different assays can be performed: one with respect to the first region and one with respect to the second region. Thus, two different sets of results can be obtained for a single sample and a single reaction phase (e.g., simultaneous reactions), and optionally a single analysis phase (e.g., detecting data from an entire reaction zone containing multiple multiplexing regions). In the aforementioned example, a single reaction phase and single analysis phase can result in a determination of whether the first and second pathogens are present in the sample. Any number of regions can be used for multiplex analysis, including any combination of different primers, probes, and/or reagents.

In some cases, different multiplex regions of a reaction zone can be structurally and/or topologically identical to one another (e.g., having passages of the same dimensions and plasmonic nanoantennae of the same composition and shapes). In such cases, the different multiplex regions may be pre-loaded with different primers, probes, and/or reagents, as disclosed herein. In other cases, however, different multiplex regions of a reaction zone can be structurally and/or topologically different form one another to effect the different multiplex analyses, such as having differently sized or shaped passages, differently sized or shaped pillars, and/or plasmonic nanoantennae having different materials or layers. Other differences can be used as well.

In some cases, a reaction zone can contain plasmonic nanocavities to facilitate quantum plasmonic PCR, as described herein. Plasmonic nanocavities can be a part of the fluid network, such as the passages, cavities, trenches, or other features of the reaction zone. Plasmonic nanocavities can be defined at least in part by walls or surfaces that are plasmonic nanoantennae. In some cases, a plasmonic nanocavity can include opposing walls or surfaces that are plasmonic nanoantennae that are separated by a distance on the order of tenths of or ones of nanometers, which can be known as a plasmonic nanogap junction. In some cases, the plasmonic nanoantennae can be separated by a gap that is at or less than approximately 10, 9, 8, 7, 6, 5, 4, 3, 2, 1, 0.9, 0.8, 0.7, 0.6, 0.5, 0.4, or 0.3 nanometers. In some cases, the plasmonic nanoantennae can be separated by a gap that approximately 2-8 nm, 3-5 nm, 2.5-4 nm, or 3-4 nm. This gap width can correspond to the width of a passageway, cavity, or other feature of the fluid network in the reaction zone, which can be considered the distance between opposing walls of the nanocavity. Plasmonic nanoantennae can be walls of the pillars that define (e.g., bound) the features of the fluid network that lie the reaction zone, which walls include plasmonic materials or have been otherwise treated to exhibit plasmonic resonance. In some cases, a plasmonic nanoantenna can include a structure (e.g., hexagonal pillar) having one or more layers including at least a layer of a plasmonic material (e.g., a plasmonic layer), such as gold or silver. The plasmonic layer can be a thin film layer (e.g., approximately 100 nm-200 nm in thickness) of the plasmonic material, although any suitable thickness can be used. In some cases, other layers, such as dielectric films (e.g., TiO₂ or other dielectric coatings), can be used underneath or over the plasmonic layer. In some cases, a polyethylene glycol (PEG) layer can be used. The polyethylene glycol layer can be an outermost layer (e.g., in contact with the fluid in the fluid network) or can at least be present over the plasmonic layer. The polyethylene glycol layer can be added through PEGylation during chip manufacturing. In some cases, a highly hydrophobic artificial surface can be used (e.g., using an outermost layer having a hydrophobic surface) in at least some portions of the fluid network to facilitate directing particles to desired locations, such as passages, cavities, and other features of the fluid network used for reacting the particles (e.g., a reaction well).

In some cases, plasmonic nanoantennae can have other shapes to allow for manipulation of optical fields as well as the concentration of electromagnetic fields. When hexagonal pillars are used, higher densities of electrons may be present at the corners between faces of the hexagonal pillars, providing high, localized plasmonic heating.

Plasmonic nanocavities can be fabricated using any suitable technique. In some cases, plasmonic nanocavities can be fabricated using silicon substrates and e-beam lithography, after which thermal oxidation of patterned silicon structures and metal deposition can occur to reduce the gap to a desired size. In some cases, plasmonic nanocavities can be formed using atomic layer lithography, extreme ultraviolet (EUV) lithography, multiple EUV lithography, or interference/holographic lithography.

A plasmonic nanoantenna can be made to include any suitable plasmonic material that exhibits plasmonic resonance, such as gold, silver, aluminum, platinum by permitting free electrons on the surface of the material to resonance (e.g., in response to light impingement). A plasmonic nanoantenna can enhance light absorption and can provide efficient local heat generation by photothermal conversion. Also, plasmon-induced electron can transfer from plasmonic nanoantennae to nearby materials, such as semiconductors, organics, polymerases, and nucleic acids. This electron transfer, as well as the plasmon resonance energy transfer, can promote enzyme activity and DNA polymerization, such as by enhancing their biochemical reactions, which can lead to an increase in amplification speed (e.g., an increase in PCR rate or a decrease in time necessary to complete PCR).

In some case, an array of plasmonic nanoantenna with a heat sink can further facilitate cooling nearby materials. The plasmonic nanoantenna can include, be coupled to, or be near heatsink materials, such as silicon or aluminum, which may facilitate cooling within the reaction zone.

Exposure of the nanoantenna to light can excite free electrons on the surface of the nanoantenna. Energy level change by electron-electron scattering can result in rapid temperature increase on the surface of the nanoantenna (e.g., rapid surface heating). The temperature can quickly equilibrate by electron-phonon coupling (e.g., lattice heating). Once the light source is turned off, the heat energy may be rapidly dissipated to the surrounding environment (e.g., heat dissipation).

In the reaction zone, reaction particles (e.g., DNA) can become located or trapped within the plasmonic nanocavities. Upon application of suitable electromagnetic radiation, such as light energy (e.g., infrared, visible, or ultraviolet), the plasmonic nanoantennae can provide localized heating of nearby fluid, reagents, and reaction particles, as well as provide quantum plasmonic resonance energy transfer to induce further heating and improved reaction speed to perform reactions (e.g., denaturing DNA or polymerization). The use of plasmonic nanoantennae to facilitate PCR can be referred to herein as quantum plasmonic PCR.

The light energy provided to the reaction zone for the quantum plasmonic PCR can be provided from any suitable light source, such as a light emitting diode (LED). In some cases, suitable LEDs can be obtained for low costs and with low power consumption, such as at or less than approximately 3 watts.

In some cases, the passages, cavities, or other features of the fluid network within the reaction zone can be configured for digital PCR (e.g., digital quantum plasmonic PCR). In such a configuration, the reaction particles can be distributed to a plurality of nanoliter cavities (e.g., passages, cavities, or other features), each of which can contain either zero or one target nucleic acid to be amplified and analyzed. In some cases, the fluid network can be configured to ensure only zero or one target nucleic acid (e.g., single strand or single double helix) can be trapped within a single cavity, however in some cases, the fluid network can be configured to ensure that approximately zero or one target nucleic acid (e.g., zero, one, or possibly a small additional number of nucleic acids) will be trapped within the single cavity. Quantum plasmonic PCR can be conducted in each of these cavities simultaneously. When analyzed using any suitable detection technique, cavities that initially contained a nucleic acid will be detected (e.g., due to the presence of the nucleic acid and the numerous copies made during amplification), whereas cavities that initially did not contain a nucleic acid will not be detected (e.g., will be detected as empty). By counting the number of cavities containing the target nucleic acid, definite or substantially definite amounts (e.g., numbers or percentages) of the target nucleic acid in the original sample can be determined. Thus, digital quantum plasmonic PCR can achieve calibration-free absolute quantification of molecules. This quantification can be useful in many instances, such as when comparing against clinical cut-off values.

In some cases, a lysing zone, a reaction zone, and/or a combination lysing and reaction zone can be used to trap, lyse, and react exosomes. In this zone, the gaps between pillars (e.g., dimension of the passages) can be between approximately 10-100 nanometers, such as at least approximately 5 nm, 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, or 95 nm. In some cases, other sizes can be used, such as those above 100 nm or below 10 nm, depending on the size of exosome to be trapped.

As exosomes pass through the gaps between the pillars, they can be optically trapped in place through the application of light onto the plasmonic surfaces of the pillars. Light reaching the plasmonic surfaces can generate surface plasmons, which can couple together with neighboring surface plasmons. At positions where the gap between adjacent pillars is close (e.g., at plasmonic nanocavities), the interaction between neighboring surface plasmons can generate a plasmonic field across the gap. Exosomes within or passing into the plasmonic field may be trapped due to the interactions with the plasmonic field. Thus, the application of light can be used to optically trap exosomes or other particles in place at desired locations within the chip. Further, the use of plasmonic nanocavities can permit exosomes and other particles to be trapped using light without the need for complex and highly focused illumination systems, such as focused lasers. Rather, the diffuse light of an LED can provide the necessary optical energy that result in the trapping of the exosome or other particle.

Once the exosome is trapped in a plasmonic nanocavity, application of further light (e.g., more intense light) can be used to generate heat within the plasmonic nanocavity and heat up the exosome. The application of heat can be precisely controlled to reach a point where the exosomes begins to lyse. Once lysing occurs, the light energy can be removed and the exosomes can be returned to a lower temperature.

After lysing, application of light in controlled patterns or cycles can be used to perform reactions, such as PCR, as described herein. Since the exosomes have been lysed, nucleic acids may be able to exit the exosomes and/or reagents may be able to enter the exosome, thus enabling reagent-based reactions that may not have been possible before lysing. In this fashion, reactions can be performed on exosomes without the need for chemical or electrochemical lysing.

In some cases, reacted materials, such as amplified nucleic acids, can be detected while within the diagnostic chip, such as within the same passages, cavities, and/or other features of the fluid network used to react those materials. Reacted materials can include any material that has been subjected to reaction within the reaction zone, such as amplified DNA.

Reacted materials can be detected and/or measured in any suitable technique. Reacted materials can be detected optically, electrically (e.g., via cyclic voltammetry), or otherwise (e.g., via radiolabels, non-optical electromagnetic radiation, or the like). A suitable sensor can be used based on the detection technique. For example, optical detection can make use of an optical sensor, such as an image sensor (e.g., camera).

In some cases, detection can include providing incident light to invoke a response, such as a fluorescent response or other emitted radiation in response to the incident light. For example, optical detection can include detection of quenching dips in the spectrum of optical radiation emitted during plasmonic resonance electron transfer after a corresponding plasmonic nanoantenna has been irradiated with light energy, as described herein. In some cases, plasmonic resonance electron transfer can invoke or facilitate fluorescence, such as fluorescence of fluorescent labels. Any suitable light source can be used to invoke a detectable response (e.g., fluorescent response), however in some cases the light source for invoking a detectable response can be the same light source used to carry out the reaction (e.g., PCR). For example, a single LED or set of LEDs can be used to not only carry out a PCR reaction during a reaction phase, but also to invoke a detectable response during a detection phase. Any suitable light source can be used, although in some cases, it can be desirable to use a LED capable of providing light energy to an entire reaction zone simultaneously, rather than a laser light source, which may be limited to providing light energy to portions of the reaction zone at a time (e.g., due to a narrow beam diameter). In some cases, a light source configured to illuminate an entire reaction zone simultaneously can be beneficial for multiplex assays.

Diagnostic chips can be processed (e.g., reacted and/or analyzed) on any suitable device (e.g., processing device). In some cases, a processing device can be a floor-based, workbench-based, mobile, or portable device. In some cases, a processing device can process one chip at a time, or multiple chips at a time. A processing device can be couplable via wired (e.g., universal serial bus) or wireless (e.g., Bluetooth or WiFi) connection to a computer, tablet, smartphone, or other computing device. In some cases, a processing device can include an integrated computer or computing device. In some cases, a processing device can couple to a network, such as a local network or a wide area network (e.g., the Internet).

The chip can be placed in, on, or under a processing device during processing. In some cases, the chip can be placed in a receptacle of the processing device. The receptacle can fully or partially receive the chip during processing. The processing device can control application of light form a light source in a desired pattern for performing the desired reaction (e.g., cycles of PCR). Light can be applied from one or more integrated light sources. In some cases, a light coupler can be used to direct light from the light source onto the chip. Light can be directed through a window or light pipe of the chip and onto the plasmonic nanoantennae of the chip. The light source can be any suitable light source, such as an LED. Any suitable wavelength of light can be used, such as infrared, visible, or ultraviolet. The wavelength of light can be tuned to the plasmonic nanocavity to achieve efficient results.

In some cases, the processing device can further include a detector for detecting and/or measuring data from the processing device, such as fluorescence or other emissions. Any suitable detector can be used, such as a camera or other imaging sensor (e.g., metal-oxide semiconductor sensor) to detect fluorescence from the chip. In some case, a light source can induce fluorescence or other detectable emissions in the reaction zone of the chip. In some cases, a single light source (e.g., single LED or LED array) can be used to both perform reactions and detection, although that need not be the case. In some cases, the processing device can include supplemental optical equipment, such as lenses and couplers.

In some cases, the processing device can further include a temperature sensor for monitoring a temperature of the chip, however that need not be the case. In some cases, application of light energy during a reaction can be performed based on a preset plan designed to achieve desired temperature cycling. In some cases, the application of light energy can be based, at least in part, on feedback from a temperature sensor. In some cases, the application of light energy can be based, at least in part, on a thermal model of the reaction zone.

The processing device can perform analytics on the image data detected by the imaging sensor, or can offload the image data to another computing device, such as a computer, tablet, smartphone, or server. In some cases, additional metadata can be provided, such as chip serial number, patient identification, location information (e.g., Global Positioning System information), assay information (e.g., sample source location or type of sample), or any other such data. Metadata can include automatically generated data (e.g., a timestamp automatically generated during processing) or manually entered data. Manually entered data can be entered using any suitable input device, such as a keyboard, a touchscreen, a camera (e.g., to read barcodes or take photos of a patient or sample site), or other such input devices. Input devices can be integrated into the processing device (e.g., a touchscreen), removably coupled to the processing device (e.g., a removable keyboard), or otherwise networked to the processing device (e.g., input devices on a smartphone). In some cases, offloading data (e.g., image data and metadata) to another computing device can include using a relay device (e.g., a smartphone) to relay data from the processing device to the computing device (e.g., a cloud-based server).

In some cases, the processing device or a computing device coupled thereto (e.g., coupled directly or networked) can perform initial processing on the image data. Initial processing can include performing one or more image manipulations (e.g., image rectification, normalization, and masking) as well as optionally analyzing the image data. Analyzing the image data can include determining where fluorescence or other emissions were detected on the chip. In some cases, these locations can be associated with particular assays of a multiplex test, although that need not be the case. Analysis of the image data can result in summary data (e.g., total count of target nucleic acids in a digital assay) and/or diagnostic data (e.g., an inference that a particular pathogen is present in the sample). In some cases, analyzing image data can result in structured data based on particular features identified in or inferred from the image data. Analysis can include leveraging a model, such as a machine-trained model (e.g., a deep neural network). In this fashion, a user may be able to obtain initial results when using the processing device, while the actual image data can be transmitted to a server for further, and potentially more accurate, analysis, such as using more computationally expensive modeling techniques and/or more extensive models. In some cases, this analysis is performed on a server and results can be sent back to the processing device or other computing device (e.g., smartphone) for presentation to a user or patient. Data presented to a user (e.g., clinician) or patient can be presented in a user-friendly format. The user-friendly format can be based on features that have been identified in the image data or inferences made after application of the image data to a model. The user-friendly format can include explanation information for explaining why particular features were identified and how certain inferences were made.

Models used for analysis can be trained in any suitable way, including random forests, support vector machines, and Bayesian networks. Further, deep learning and deep neural networks can be used to analyze data to construct and/or improve a model. A deep neural network can be trained in any suitable fashion, such as through supervised learning. Since chip topology may vary slightly between fabrications, exact locations of individual passages, cavities, or other features of the fluid network in which reactions have taken place may not necessarily be located in the sample place with respect to the imaging sensor between different chips. Therefore, deep neural networks (e.g., convolutional neural networks) can be trained to process the image data and identify pixels representative of individual passages, cavities, or other features of the fluid network. Further, denoising autoencoders can be used to facilitate estimating reading from uncertain pixel data. Restricted Boltzmann Machines can be used to reduce the high dimensionality in the data. Metadata used to train the models can facilitate accounting for certain variations in image data, such as variations due to disease heterogeneity, sampling site (e.g., finger prick or skin swab), time of day sample was obtained, or other reasons. In some cases, change detection techniques can be used to determine when a machine-learning model is to be retrained, to mitigate data drift and the appearance of new diseases. In some cases, incoming data (e.g., image data and metadata) can be used to further improve existing models.

Use of a diagnostic chip according to certain aspects and features of the present disclosure can include receiving a sample, preparing the sample for reaction (e.g., separation and lysis), and reacting the sample (e.g., PCR). The sample can be received at the sample input zone using any suitable technique, such as those described above. The chip can be reacted using a processing device, as disclosed herein. In some cases, the processing device can additional detect and/or analyze the chip, such as to generate image data and/or diagnostic results.

Sample preparation can include multiple aspects, such as separation and lysis. Separation can include removing cellular components and debris from sample fluid. Efficient and effective debris removal can be important to improve the analytical sensitivity and specificity of the PCR and subsequent analysis. Further, separation can include removing or eliminating inhibitors to PCR. Lysis, which can occur after separation, can facilitate extracting nucleic acids from lysable particles. For example, pathogen analysis may require lying of the pathogens to extract the nucleic acids which will be amplified using PCR. While separation and lysis may be time-consuming and labor intensive in conventional PCR techniques (e.g., requiring manual preparation steps, centrifugation, lysing, and filtration), certain aspects and features of the present disclosure can achieve suitable, comparable, or even improved results over conventional PCR techniques. Certain aspects and features of the present disclosure enable on-chip separation and lysing in an ultrafast process. Further, the use of self-powered capillary action to drive the separation any lysing process in certain aspects of the present disclosure avoids the need for expensive pumps with high power consumption. Further, the use of a functional gradient as disclosed herein can permit rapid separation without the need for filters, which can clog and cause hemolysis or other undesirable damage to particles in the same. Avoiding hemolysis can be important to producing efficient, accurate, and reliable PCR and subsequent analysis, but can be difficult to achieve without drastically slowing the separation process, which may also require the use of additional treatment to prevent coagulation. Certain aspects and features of the present disclosure, however, can provide rapid separation and high-throughput separation of a sample with minimal or substantially no risk of hemolysis.

Additionally, the self-powered capillary action disclosed herein can provide opportunities to improve miniaturization, as reliance on external equipment, such as pumps, amplifiers, acoustic generators, motors, and other such equipment can be minimized or eliminated.

Lysing can occur through any suitable technique, although improved results can be achieved through the use of on-chip lysis via electrochemical generation of hydroxide, as described herein.

Reacting a sample can include performing a reaction by heating and cooling material (e.g., reaction particles and reagents) in one or more controlled cycles, such as PCR cycles. Reacting a sample can include taking advantage of efficient photothermal heating via quantum plasmonic resonance energy transfer.

In one example, PCR can be achieved by raising the temperature of a nucleic acid strand (e.g., DNA strand) to a desaturation temperature (e.g., approximately 95 degrees C.) to allow the nucleic acid strand to denature into two template strands. Then, the temperature can be lowered to an annealing temperature (e.g., approximately 50-65 degrees C.) to enable primers to attach to the individual template strands. The metal, the temperature can be raised to an extension temperature or polymerization temperature (e.g., approximately 72 degrees C.) to permit a new strand of nucleic acid to be generated by the polymerase enzyme. These temperature changes can be repeated numerous times (e.g., approximately 30-45 times). Each cycle can double the number of copies of nucleic acid strands. The process of applying light to perform a reaction (e.g., PCR), regardless of the number of cycles necessary, can be considered a reaction phase.

As disclosed herein, the use of plasmonic materials on the walls of passages and other features of a fluid network enable the benefits of photothermal heating and quantum plasmonic resonance energy transfer to be consistent and reproducibly utilized. Since the plasmonic material is fixed with respect to the fluid network (e.g., fixed as part of the surface of the pillars defining walls of the fluid network), there is no opportunity for the plasmonic materials to move freely and potentially collect in higher concentrations in some regions and lower concentrations in other regions, which may negatively affect reactions and analysis.

Photothermal heating can include any process of converting light energy into heat energy. In some cases, photothermal heating can be achieved at least in part by light interacting with plasmonic materials of the walls of the fluid network located in the reaction zone to generate thermal energy in and adjacent to the plasmonic material. Impinging light energy can be adsorbed by the plasmonic layer to form plasmons (e.g., surface plasmons), which, in turn, decay, generating heat. Once light impingement ceases (e.g., the light is turned off), the generation and decay of plasmons ceases and thus cooling is achieved. In some cases, the plasmonic material can further provide cooling by directing heat away from the nearby particles via thermal conduction. In some cases, substrate materials with high thermal conductivity (e.g., silicon) can improve cooling. Further, the use of many plasmonic nanocavities can increase photothermal efficiency by increasing light absorption.

Light can also directly impinge any fluid or materials in the fluid network to achieve some degree of photothermal heating. In some cases, the walls of the fluid network, including walls incorporating plasmonic material, can include thin films in which impinging light may undergo total internal reflection, thus maximizing the amount of energy supplied by a pulse of light.

In some cases, heating rates of at least approximately 10, 11, 12, 13, 14, or 15 degrees C. per second can be achieved, as well as cooling rates of at least approximately 5, 6, 7, or 8 degrees C. per second, although other rates may be achieved.

In addition to photothermal effects, quantum plasmonic resonance electron transfer between plasmonic materials (e.g., in plasmonic nanocavities) and polymerase and/or nucleic acids can further enhance the rate of reaction (e.g., PCR rate).

Quantum plasmonic resonance energy transfer can be used to speed up reactions in the reaction zone. Plasmonic resonance energy transfer can include using light energy to induce the transfer of electrons between plasmonic structures (i.e. optical antennas) and enzymes and/or nucleic acids. For example, enzymes and nucleic acids can act as electron acceptors, while plasmonic structures can act as electron donors. The transfer of electronics can invoke oxidation-reduction reactions of nucleic acids and polymerase within a plasmonic nanocavity. The transfer of electrons between the plasmonic nanoantennae and the nucleic acids with polymerize enzymes can provide a catalytic effect to PCR. In some cases, the use of quantum plasmonic resonance energy transfer can be leveraged to improve kinetics, specificity, and/or detection limits for PCR, as well as other improvements. Thus, in addition to providing photothermal heating, the application of light energy to diagnostic chips disclosed herein can provide further benefits to the reactions taking place within the chips.

Nanoantenna sensitivity can be tuned by designing nanocavities with different aspect ratios and gap distances. These aspect ratios and gap distances can correspond to the cross section of a passageway, cavity, or other feature of the fluid network in the reaction zone. Adjustments to this cross section (e.g., to the aspect ratio and/or gap distance of the nanocavity) can alter how electrons are transferred between the plasmonic nanocavity and the nucleic acids and/or enzymes contained therein.

Surface plasmonic resonance can include the collective oscillation of confined free conduction electrons on the surface of metals, such as gold and silver, at specific frequencies in response to impinging light. In some cases, plasmonic materials of suitable sizes and geometries can be considered plasmonic nanoantenna, as they can serve to emit a specific oscillating frequency in response to received light due to surface plasmonic resonance. In some cases, this oscillating frequency can be detected using an external detector. The detected signals from plasmonic nanoantennae can be compared with other signals, such as previously detected or expected signals to make an inference about the condition or position of the plasmonic nanoantenna, including the presence of materials coupled to or adjacent the plasmonic nanoantenna, which may induce variations in signals output from the plasmonic nanoantenna in response to light. In some cases, plasmonic material can become plasmonically coupled to a material (e.g., a biomolecule, a reagent, or a nucleic acid) through plasmonic coupling, in which case light energy impinging the plasmonic material can result in transfer of energy from the plasmonic material to the coupled material. In some cases, the resonant frequency of a plasmonic nanoantenna may be tuned to match, overlap, or otherwise correspond to an absorption peak of a coupled biomolecule (e.g., a molecular electronic transition frequency of the coupled biomolecule). In such cases, energy transfer from the plasmonic nanoantenna to the biomolecule can be detected and/or visualized by measuring and/or depicting a scattering spectrum of the plasmonic nanoantenna. The scattering spectrum can be detected by optical spectroscopy or other suitable techniques. At the various absorption peaks of the coupled biomolecule, the scattering spectrum of the plasmonic nanoantenna will include detectable, quantized quenching dips. Thus, the use of plasmonic nanoantennae can provide for the detection of molecules of a sample at single molecule-level sensitivity by detecting the scattering spectrum of plasmonic nanoantennae in response to impinged light. Those plasmonic nanoantennae that are plasmonically coupled to nearby biomolecules will emit identifiable scattering spectrums, which can each be correlated to the specific biomolecule that is coupled to a particular plasmonic nanoantenna through identification of the detected quenching dips. Since different molecules have different fingerprints of electron transition frequencies (e.g., absorption peaks), different molecules will induce different quenching dips when plasmonically coupled to a plasmonic nanoantennae. Such a detection system based on plasmonic resonance energy transfer can enable quantitative and long-term dynamic imaging of biomolecules without the drawbacks of photo bleaching and blinking inherent to other imaging techniques, such as Förster resonance energy transfer. Further, a detection system based on plasmonic resonance energy transfer can have a sensitivity many orders of magnitude higher than other techniques, including quantum dot imaging and conventional colorimetric organic dye detection. In some cases, detection of nucleotides can be achieved using plasmonic resonance energy transfer techniques, as described herein, without the need for nucleotide labeling.

Certain aspects and features of the present disclosure, including Quantum Plasmonic PCR, can enable copying of sufficient nucleic acids for detection in under approximately three minutes (e.g., approximately 30 cycles) with sensitive of less than approximately 10 copies from a 5 microliter sample. In some cases, nucleic acid concentrations as low as two copies per microliter of a sample can be successfully amplified and detected. In some cases, ribonucleic acid (RNA) at low concentrations of approximately 10,000 copies per milliliter can be successfully amplified and detected. In some cases, these or similar results can be achieved from sample through amplification with low power consumption, such as approximately 3 Watts (e.g., at or under approximately 2, 3, 4, 5, or 6 Watts). Certain aspects and features of the present disclosure can work with very small volumes (e.g., on the cubic nanometer scale), to achieve rapid heating and cooling cycles. Ultrafast photothermal PCR cycling can be achieved using low power from a light source without relying on high-power heating elements (e.g., resistive heaters).

Certain aspects and features of the present disclosure, including combinations of an integrated functional gradient structure, self-powered pumping, integrated lysing, and Quantum Plasmonic PCR, can achieve ultrafast results, such as a sample-to-answer time (e.g., time between supplying the sample to the sample input zone and receiving a detectable result) of at or less than approximately 4, 5, 6, 7, 8, 9, 10, 11, 12, 13, 14, or 15 minutes.

The diagnostic chip disclosed herein can be useful for performing any suitable reactions, including PCR. In some cases, certain aspects and features of the present disclosure can be especially useful for performing rapid PCR to screen for bacteria or viruses. The mobile nature and high speed of the diagnostic chip and processing device can enable on-the-fly diagnostics in many scenarios, such as hospital rooms, physician offices, remote cities and villages, and temporary triage facilities, among others. Further, the mobile nature of the diagnostic chip and processing device permit PCR analysis from sampling to results entirely at a patient site, rather than requiring transportation of sampling equipment, samples, results, and the like between various sites (e.g., patient sites, processing sites, laboratories, analysis sites, and the like).

Certain aspects and features of the present disclosure enable fast PCR (e.g., faster or much faster than traditional PCR techniques), which can reduce the discovery time for biological mechanisms, signalling pathways, biomarkers, and drugs, as well as mitigate the spread of infectious disease. For example, once a disease is accurately and quickly identified, appropriate therapeutic treatments can be determined and applied.

By way of further example, methicillin resistant Staphylococcus aureus (MRSA) is a serious worldwide threat, being a pathogen that is resistant to many drug classes and is carried by approximately 2% of the worldwide population. Active surveillance and early isolation of patients with MRSA is important to minimizing the risk of infections, especially in healthcare facilities, where risk of passing on the infection is high and where the number of isolation rooms may be limited. Reliance on standard MRSA culture-based analysis can require more than two days before a result is obtained, which may result in improper occupation of isolation rooms for patients without MRSA and loss of revenue from bed closures. Reliance on standard PCR-based MRSA analysis can be costly, require specialized equipment and laboratory space, and can require specialized lab technicians, as well as still requiring several hours before a result can be obtained. Certain aspects of the present disclosure can permit screening of MRSA by its genetic elements in only a few minutes (e.g., less than 5 minutes) without the need for the same amount of equipment and laboratory space. Further, the portable nature of certain aspects of the present disclosure can permit patients to be tested prior to entering a healthcare facility, such as en route in an ambulance, thus permitting the staff to isolate the patient immediately upon admittance (e.g., have the patient treated in an isolation emergency room).

Certain aspects of the present disclosure, as described herein, can be further used to facilitate observation and control of electron transfer within biological systems. For example, the nanoantennae described herein can be used for imaging electron transfer of mitochondrial cytochrome c from death (e.g., apoptosis) to life. In another example, nanoantennae described herein can be used to screen brain organoids in vitro for the presence of various drugs based on the principles of QBET. In another example, a nanoantenna described herein can be integrated into an implantable device and used to perform interventions based on the principles of QBET, such as to serve as molecular pacemakers.

In one example, QBET principles can be used to perform interventions related to certain neurodegenerative disorders and brain cancers. Lowered ATP production, altered glucose uptake, and increased Reactive Oxygen Species (ROS) production can be common hallmarks of many neurodegenerative disorders and brain cancers. A nanoantenna can be used to modulate mitochondrial oxidative phosphorylation enzymes, such as using near-infrared-based, electromagnetic, or other QBET methodologies. This modulation of the mitochondrial oxidative phosphorylation enzymes can facilitate restoring the equilibrium necessary for normal cellular functioning, and may slow the progression of cancers and neurodegeneration.

In some cases, a magnetoplasmonic nanoantenna (e.g., a nanocrescent antenna) can be used for imaging and/or modulating the electron transfer of mitochondrial cytochromes. In some cases, nanoplasmonic antennae can be used to control an electromagnetic field suitable to modulate Channelrhodopsin. Such nanoantennae can be used to control excitable cells and/or intracellular redox biochemistry.

A nanocrescent antennae can be engineered to achieve desired responses. For example, the radius of the cavity opening and/or the cavity depth can be selected to achieve selective wavelength and field enhancement. In some cases, a nanocrescent antenna can comprise integrated plasmonic and magnetic layers. Electromagnetic excitations coupled to surface plsamon waves on metal-dielectric interfaces or localized on metallic nanostructures can enable the confinement of light to scales far below that of conventional optics.

In some cases, a diagnostic chip can comprise nanoantennae and can leverage QBET principles to detect biomarkers representing key systems (e.g., monoamine, neutrophic, excitotoxicity, inflammatory, and cellular-metabolic systems) in certain disorders (e.g., neurological disorders). In some cases, a diagnostic chip can leverage the principles of quantum plasmon resonance energy transfer and QBET, such as to perform PCR and protein and cytokine detection. In some cases, QBET can be used to increase the sensitivity and detection limits of PCR as otherwise described herein.

In some cases, specific targeting and signal amplification with functionalized nanoplasmonic antenna probes can be used to perform detection of biomolecules (e.g., mRNA, miRNA) in various environments, such as native environments in vivo.

In some cases, magnetoplasmonic nanoantennae can be cultured within organoids (e.g., mini-brain organoids on-chip) or positioned proximate the organoid to achieve control and/or monitoring of various relevant aspects, such as electrophysiological dynamics, ion channel activity, neuron action potentials, and organoid growth. In some cases, nanotantennae modulation of ion channels of a mini-brain organoid can enable tuning of neuronal activity that may be detectable as a change in electroencephalogram (EEG). Such nanoantennae incorporated with organoids can be used to facilitate sensing and controlling aspects of the organoid.

In some cases, nanoantenna interventions can be used to control synaptic ion channels, thus enabling modulation of neuronal activity. Nanoantenna interventions can reduce or eliminate the need for drug-based interventions, such as those relying on small molecule drugs, or invasive optogenetic or physical probe interventions. Nanoantennae can be incorporated into a neurological pacemaker device capable of modulating neuronal activity when a need for such modulation is detected. Unlike small molecule drugs, nanoantennae neuronal modulation can be achieved with time-precise phasic activation and deactivation of ion channels.

These illustrative examples are given to introduce the reader to the general subject matter discussed here and are not intended to limit the scope of the disclosed concepts. The following sections describe various additional features and examples with reference to the drawings in which like numerals indicate like elements, and directional descriptions are used to describe the illustrative embodiments but, like the illustrative embodiments, should not be used to limit the present disclosure. The elements included in the illustrations herein may not be drawn to scale.

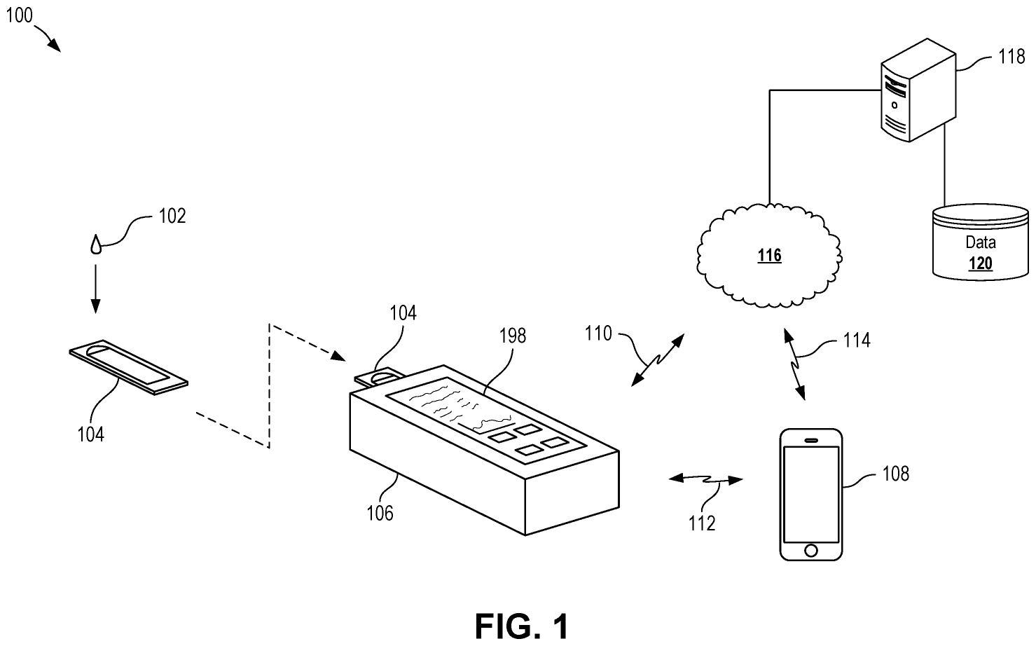

FIG. 1 is a schematic diagram of a plasmonic PCR system 100 according to certain aspects of the present disclosure. A diagnostic chip 104 can receive a sample 102. The sample 102 can be fluid (e.g., a fluid or particles entrained in a fluid), such as breath condensate, blood, or a skin swab sample. The sample 102 can be placed within a input port of the diagnostic chip with a purpose-built tool (e.g., a syringe or applicator) or by simply dropping the sample into the input port (e.g., dropping a blood sample into the input port). In some cases, the diagnostic chip 104 can include a build-in sample extraction device, such as a lancet designed to elicit a blood sample upon pressing a finger or thumb against the sample input port.

After receiving a sample 102, the diagnostic chip 104 can be left to perform initial processing (e.g., filtration) or can be immediately placed within a processing device 106. The processing device 106, or reader, can include a slot for receiving the diagnostic chip 104. The diagnostic chip 104 can be placed entirely within the processing device 106, partially within the processing device 106, or adjacent to the processing device 106 (e.g., above or below). The processing device 106 can provide light energy to the diagnostic chip 104, such as using an LED, to induce plasmonic activity in the diagnostic chip 104 to facilitate reactions and/or other functions of the diagnostic chip 104. The processing device 106 can also optionally provide electricity, pressure, a vacuum, or other forces to the diagnostic chip 104 to facilitate operation of the diagnostic chip 104 of the plasmonic PCR system 100 as a whole.

The processing device 106 can record information about the diagnostic chip 104, including results from processing and reacting the sample 102 within the diagnostic chip 104. For example, in cases where the sample 102 is a blood sample, PCR can be performed on the DNA from the sample 102 with the aid of plasmonic resonance energy transfer controlled through the application of light energy from the processing device 106, then the results of the PCR can be imaged using an imaging device within the processing device 106.

In some cases, the processing device 106 can perform some or all processing on the recorded data (e.g., image). In other cases, raw or semi-processed data can be transmitted to other computing devices for further processing and/or storage.

The processing device 106 can send signals 112 to and/or from a computing device 108, such as a smartphone, laptop, desktop computer, tablet, or other such device. The computing device 108 can interact with the processing device 106 to receive raw, semi-processed, or processed data. In some cases, some or all of the data processing can be offloaded from the processing device 106 to the computing device.

The processing device 106 and/or the computing device 108 can send signals 110 and/or signals 114, respectively, over a network 116 (e.g., a local area network, a cloud network, or the Internet) to communicate with a server 118. The server 118 can receive data from the processing device 106 and/or computing device 108 to perform further processing, such as image analysis, deep neural network analysis, model training, and other processing.

The server 118 can include one or more computing devices. The server 118 can be coupled to a data storage 120 to store data received from the processing device 106 or computing device 108, as well as data processed by the server 118 (e.g., results from processing data).

Processed data can include image files, inferred diagnoses, detected traits, or other such information. Processed data can be displayed in a user-friendly format through the processing device 106, a computing device 108, or other such device. The processing device 106 can include an interface 198 for interacting with the processing device 106, including providing feedback to a user and/or receiving inputs from a user.

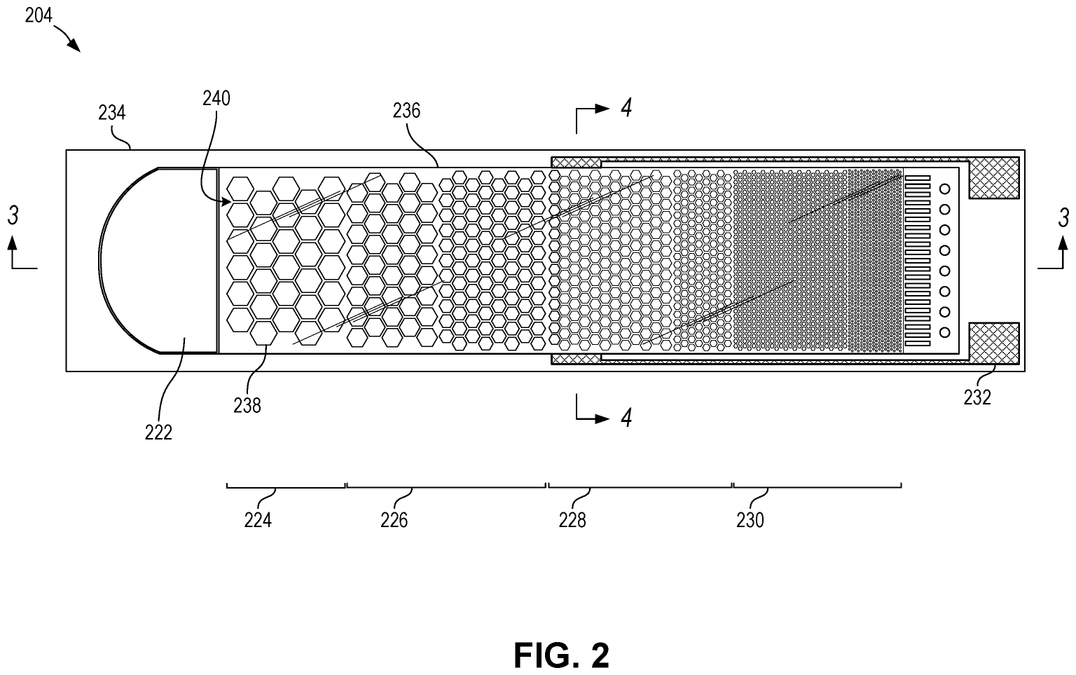

FIG. 2 is a top view of a diagnostic chip 204 according to certain aspects of the present disclosure. The diagnostic chip 204 can include a substrate 234 upon which or into which passages 240 are formed. The passages 240 can be defined in part by the walls of pillars 238, such as hexagonal pillars. The passages 240 and pillars 238 can define a fluid network through the diagnostic chip 204, starting at a sample input port 222 and extending distally along the length of the diagnostic chip 204. The widths of the passages 240 (e.g., gaps between pillars 238) can decrease in size moving away from the sample input port 222. The diagnostic chip 204 can be made of a transparent or translucent material in some cases. However, in some cases, the diagnostic chip 204 can include a window 236 through which light can pass into and/or out of the passages 240 of the fluid network.

The fluid network of the diagnostic chip 204 can include multiple zones capable of performing various functions. As depicted in FIG. 2, the diagnostic chip 204 includes a separation zone 224, a pumping zone 226, a lysis zone 228, and a reaction zone 230. In some cases, a diagnostic chip 204 can include fewer or more zones, including other zones as necessary. In some cases, the zones of a diagnostic chip 204 can be arranged sequentially, with the output of one zone flowing into the input of a subsequent zone. In some cases, however, the zones of a diagnostic chip 204 can overlap, with some passages 240 performing functions of multiple zones. For example, a single set of passages 240 can be part of both a separation zone 224 and a pumping zone 226.

The separation zone 224 can include passages 240 and cavities capable of trapping certain debris while permitting passage of desired particles. The passages 240 and cavities of the separation zone 224 can form functionally graded microfluidics capable of separating undesired particles from desired particles. In an example, when blood is provided to a separation zone 224, the blood cells of the separation zone 224 can be retained within the separation zone 224 while nucleic acids are passed further down the fluid network.

In a pumping zone 226, the passages 240 of the fluid network form capillaries capable of pumping a fluid sample from the sample input port 222 through the fluid network. The wicking capability (e.g., flow rate) of the pumping zone 226 can be tuned by adjusting the shape and/or widths of the passages 240 within the pumping zone 226.

In a lysis zone 228, exosomes or other lysable particles can be lysed through chemical, electrochemical, or other techniques. In some cases, the lysis zone can include pre-loaded materials, such as lysing agents, however that need not be the case. As depicted in FIG. 2, a set of electrodes 232 can supply electrical current to the lysis zone 228. The electrical current can induce electrochemical lysis, such as through the generation of hydroxide within the lysis zone 228. Though lysis, desired particles can be released and flow further down the fluid network and into the reaction zone 230.

In a reaction zone 230, particles to be reacted can be collected within plasmonic nanocavities. Portions of the fluid network, such as portions of the passages 240 within the reaction zone 340 can form the plasmonic nanocavities. At least within the reaction zone 340, and possibly within other zones, the walls of the passages 240 (e.g., surfaces of the pillars 238) can be coated with a plasmonic material, such as gold.

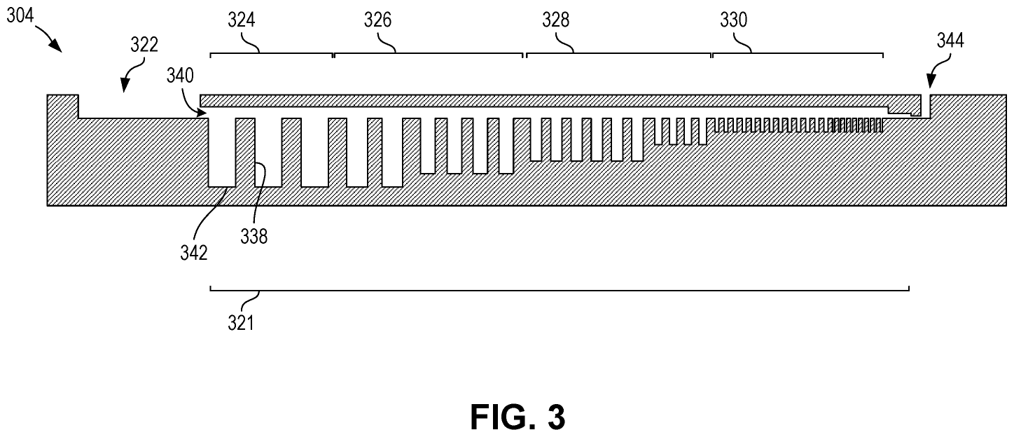

FIG. 3 is a side cross sectional view of a diagnostic chip 304 according to certain aspects of the present disclosure. The diagnostic chip 304 can be the diagnostic chip 204 taken across cross section line 3:3. The diagnostic chip 304 can include a fluid network 321 comprised of a separation zone 342, a pumping zone 326, a lysis zone 328, and a reaction zone 330. The fluid network 321 can extend distally from a sample input port 322. In some cases, the fluid network 321 can terminate in an opening 344, which can allow pressure to equalize within the fluid network 321 during capillary pumping. In some cases, however, the fluid network 321 can include a void containing a vacuum to facilitate drawing the sample into the diagnostic chip 304 without the need for an opening 344.

Fluid can enter the sample input port 322 and flow through the fluid network 321 of the diagnostic chip 304. Within the separation zone 324, cavities 342 can connect to the passages 340 to receive fluid and undesirable particles, such as blood cells or debris. The pillars 338 of the diagnostic chip 304 can act to form passages 340 and cavities 342 within the diagnostic chip 304. The cavities 342 of the fluid network 321 can have smaller heights and/or diameters progressing from the sample input port 322 distally along the fluid network 321 of the diagnostic chip 304.

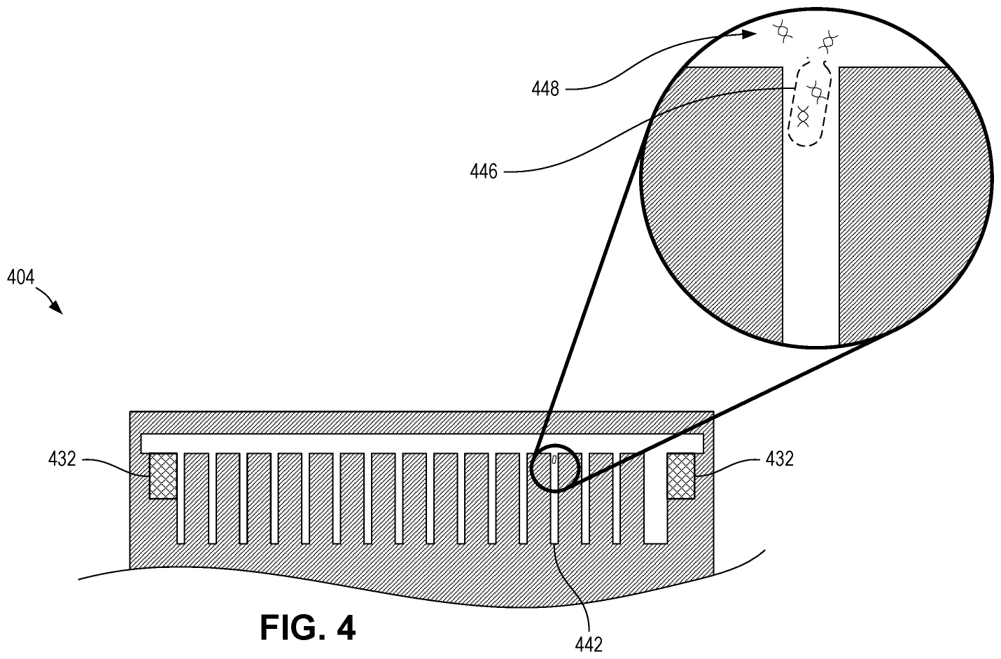

FIG. 4 is a front cross sectional view of a lysing zone of a diagnostic chip 404 according to certain aspects of the present disclosure. The diagnostic chip 404 can be the diagnostic chip 204 taken across cross section line 4:4. Within the lysis zone 428 of the diagnostic chip 404, exosomes 446 or other lysable particles can become trapped within cavities 442 of the fluid network. An electrical current can be supplied through the electrodes 432 to generate hydroxide ions within the lysis zone to induce lysis of the exosomes 446. Upon lysing of the exosomes 446, nucleic acids 448 stored within the exosomes 446 can be released to flow further down the fluid network.

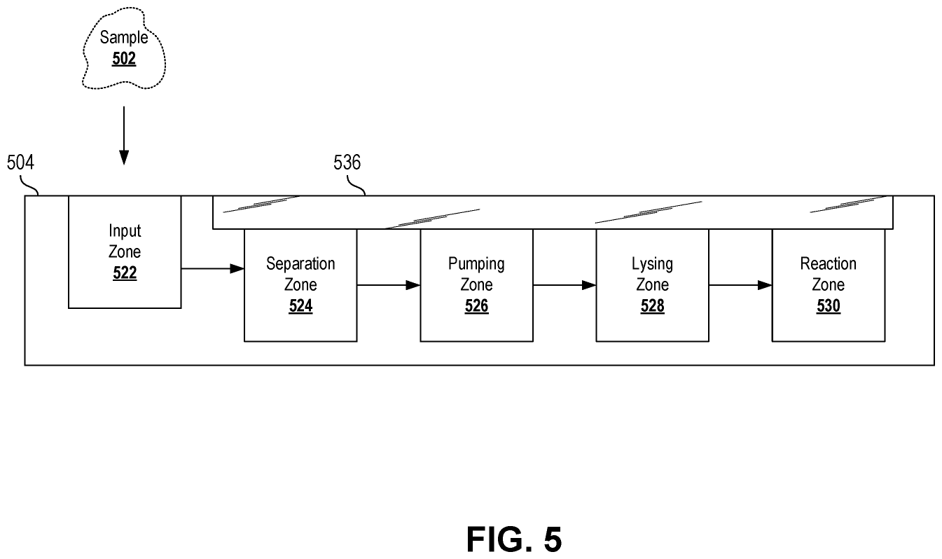

FIG. 5 is a schematic side view of an ultrafast diagnostic device 504 according to certain aspects of the present disclosure. The ultrafast diagnostic device 504 can be contained within a single housing (e.g., a diagnostic chip) or spread amongst two or more couplable housings.

A sample 502 can be received at an input zone 522. The input zone 522 can include a sample input port or other feature for receiving and/or collecting a sample 502. In some cases, the input zone 552 can include a lancet for initiating a finger prick to receive a blood sample. The input zone 522 can provide the sample to the separation zone 524.

The separation zone 524 can receive the sample from the input zone 522 and separate undesirable particles from desirable particles. The undesirable particles can be retained within the separation zone 524 and the desirable particles can be passed further along the fluid network. In some cases, the separation zone 524 can pass desirable particles through a pumping zone 526, although that need not be the case, and the pumping zone 526 may not be included in the ultrafast diagnostic device 504 or may be located elsewhere within the ultrafast diagnostic device. The pumping zone 526 can use capillary action or other force to pump the sample 502 through the fluid network.

The lysing zone 528 can receive particles from a separation zone 524, pumping zone 526, or other zone. Within the lysing zone 528 lysable particles can be captured and lysed, releasing desired particles within. Desired particles from the lysing zone 528 can be passed to a reaction zone 530.

Desired particles can be received in the reaction zone 530 from the lysing zone 538 or another zone. The desired particles can be trapped within plasmonic nanocavities of the reaction zone 530. Light can be provided through a window 536 and into the reaction zone 530 to generate surface plasmons on the plasmonic surfaces of the walls of the passages of the reaction zone (e.g., walls of the plasmonic nanocavities). In some cases, the window 536 can extend over multiple zones, although that need not be the case. In some cases, light can be used to perform or facilitate functions of zones other than the reaction zone 530, such as to induce lysis or promote pumping.

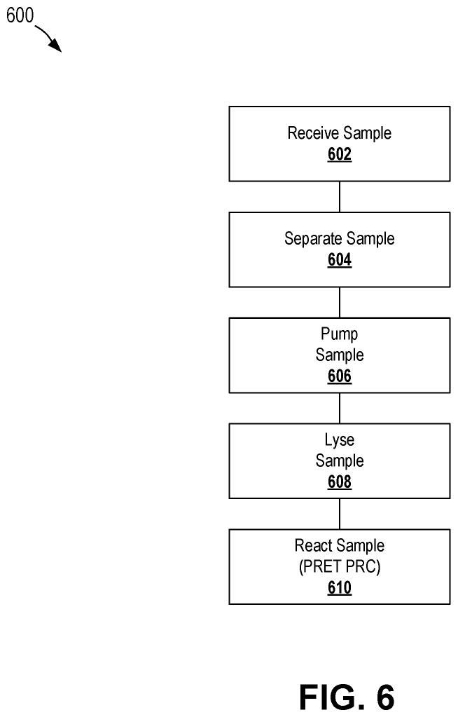

FIG. 6 is a flowchart depicting a process 600 for conducting on-chip filtering, lysing, and reacting according to certain aspects of the present disclosure. The entire process 600 can be carried out by a single ultrafast diagnostic device, such as a diagnostic chip. At block 602, a sample can be received, such as at a sample input port. Receiving the sample can include receiving a fluid sample or receiving a dry sample. If a dry sample is received, receiving the sample at block 602 can include mixing the dry sample with a carrier fluid.

At block 604, the sample can be separated into desired particles and undesired particles. The undesired particles can include debris and other particles that are to be separated prior to lysis and/or reaction. At block 606, the sample can be pumped through the fluid network of the ultrafast diagnostic device. Pumping the sample can include using capillary action to pump fluid through the fluid network.

At block 608, the sample can be lysed. Lysing the sample can include trapping a lysable particle within a passage of the fluid network of the ultrafast diagnostic device and then lysing the lysable particle through a chemical, electrochemical, or other technique. In some cases, lysing the particle can include applying an electrical current through, at, or near passages containing lysable particles to generate hydroxide ions in the presence of the lysable particles to induce lysing of the lysable particles. Lysing the lysable particles can permit desired particles within the lysable particles to be released and flow into the reaction zone.

At block 610, the sample can be reacted. Reacting the sample can include inducing a reaction in the desired particles, such as PCR. At block 610, reacting the sample can using quantum plasmonic resonance energy transfer (PRET) to facilitate the reaction. Block 610 can include applying light to the reaction zone to generate surface plasmons which can increase heat in plasmonic nanocavities and enhance the effectiveness of enzymes and/or other reagents. By controlling the pattern of light application, the temperature within plasmonic nanocavities can be regulated and cycled, such as to perform PCR. In some cases, other reactions can be performed.

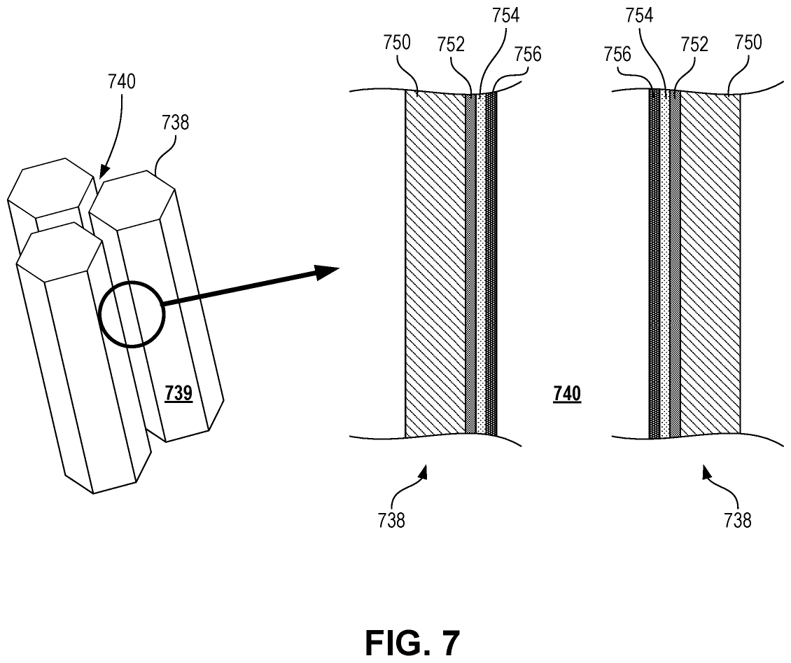

FIG. 7 is a combination axonometric diagram of a set of pillars 738 of a diagnostic chip and a schematic cross sectional diagram depicting the passageway 740 between the pillars 738 according to certain aspects of the present disclosure. The left portion of FIG. 7 is an axonometric diagram of three pillars 738, including the passages 740 between the pillars 738. Each pillar 738 can include surfaces 739 that define the walls of the passages 740.

The right portion of FIG. 7 is a schematic cross sectional diagram depicting the passage 740 between two pillars 738. Each pillar 738 can be comprised of multiple layers. In some cases, the pillar 738 can include a substrate 750 (e.g., silicon), an oxidization layer 752 (e.g., silicon dioxide), a plasmonic layer 754 (e.g., gold), and a thin film layer 756 (e.g., a dielectric layer). In some cases, more or fewer layers can be used, however a plasmonic layer 754 is to be used for the generation of surface plasmons. The plasmonic layer 754 can be any suitable plasmonic material, such as gold. The thin film layer 756 can be selected and sized to provide a high degree of total internal reflections to optimize the generation of surface plasmons.

The gap between adjacent pillars 738 can be any suitable gap width dependent upon the purpose of the passage 740. For example, a gap for trapping nucleic acids can have a relatively small gap width (e.g., approximately 3-10 nm), whereas a gap for trapping exosomes can have a larger gap width (e.g., approximately 10-40 nm). Other gap widths can be used.

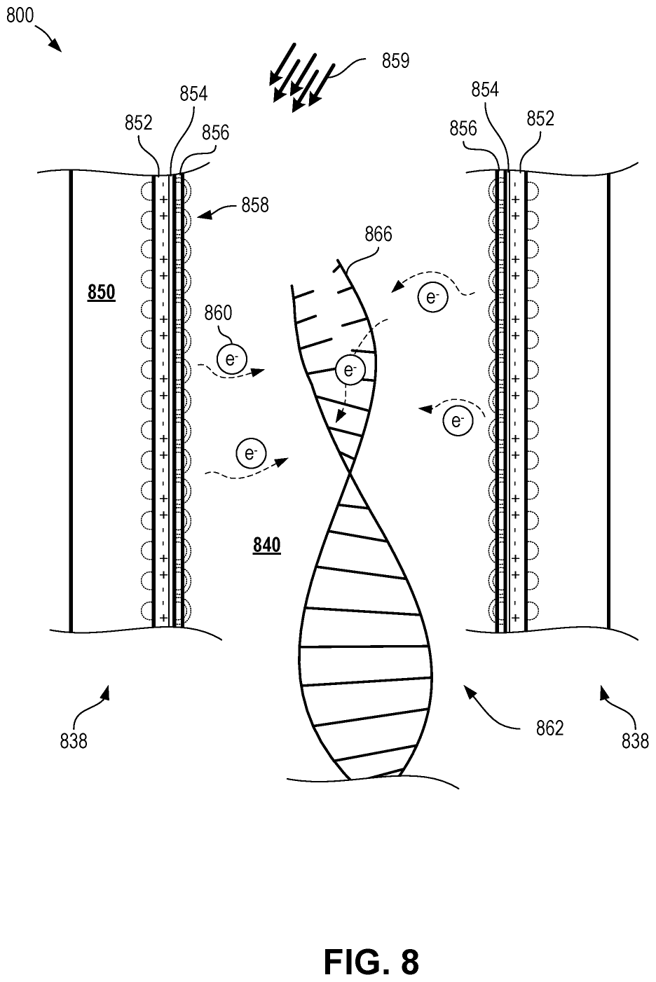

FIG. 8 is a schematic cross sectional diagram depicting plasmon-assisted denaturing of a nucleic acid 862 within a plasmonic nanocavity 800 according to certain aspects of the present disclosure. The pillars 838 can include a substrate, an oxidization layer 852, a plasmonic layer 854, and a thin film layer 856. Light energy 859 incident on the plasmonic layer 854 is inducing surface plasmons 858. The surface plasmons 858 can induce localized heating within the plasmonic nanocavity 800. Additionally, surface plasmons 858 can induce transfer of electrons 860 to and/or from the nucleic acid 862. As a result of the increased temperature and electron 860 transfer, the nucleic acid 862 can become denatured and separated into individual strands 866, which can be later replicated.

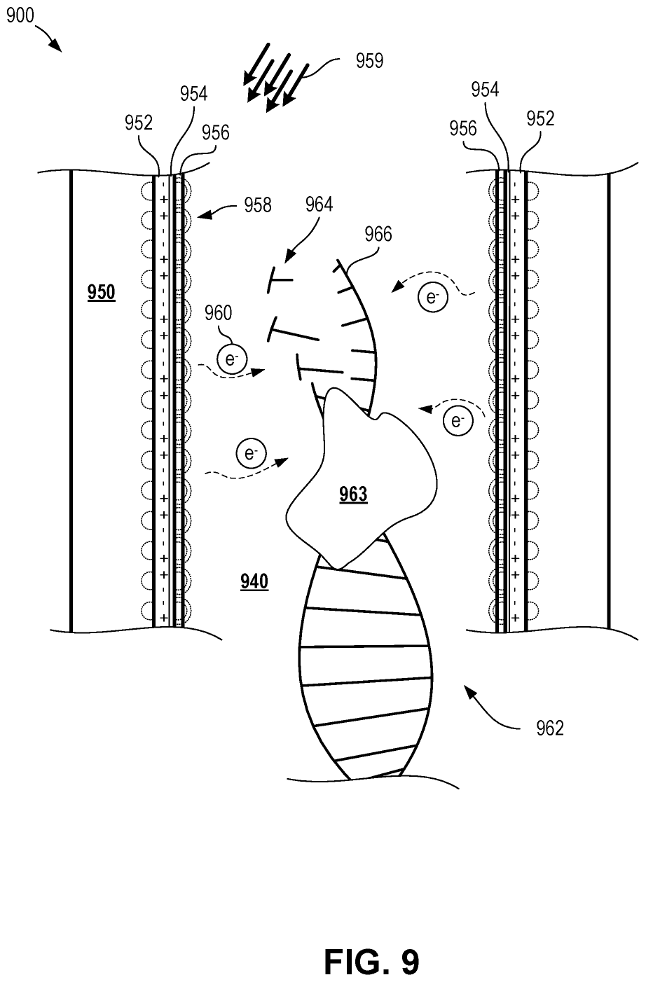

FIG. 9 is a schematic cross sectional diagram depicting plasmon-assisted elongation of a nucleic acid 962 within a plasmonic nanocavity 900 according to certain aspects of the present disclosure. The pillars 938 can include a substrate, an oxidization layer 952, a plasmonic layer 954, and a thin film layer 956. Light energy 959 incident on the plasmonic layer 954 is inducing surface plasmons 958. The surface plasmons 958 can induce localized heating within the plasmonic nanocavity 900. Additionally, surface plasmons 958 can induce transfer of electrons 960 to and/or from the nucleic acid 962 and a polymerase enzyme 963. As a result of the increased temperature and electron 960 transfer, the polymerase enzyme 963 can more effectively and/or more efficiently replicate the nucleic acid 962 by joining nucleotides to individual strands 966 of nucleotides.

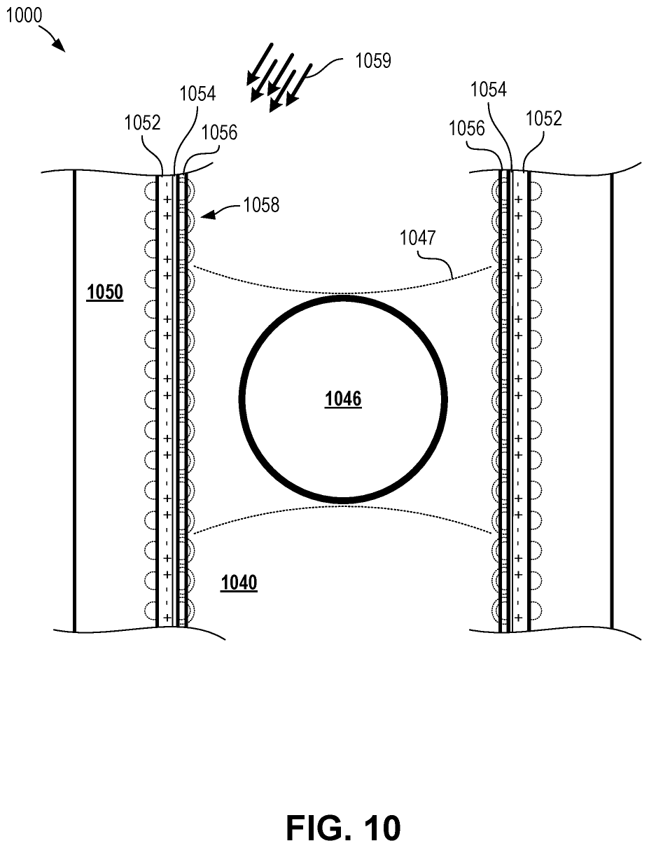

FIG. 10 is a schematic cross sectional diagram depicting plasmon-assisted trapping of an exosome 1046 within a plasmonic nanocavity 900 according to certain aspects of the present disclosure. The pillars 1038 can include a substrate, an oxidization layer 1052, a plasmonic layer 1054, and a thin film layer 1056. Light energy 1059 incident on the plasmonic layer 1054 is inducing surface plasmons 1058. The surface plasmons 1058 can interact with neighboring surface plasmons 1058 to generate a plasmonic field 1047 within the plasmonic nanocavity 1000. The plasmonic field 1047 can trap exosomes 1046 within the plasmonic nanocavity 1000.

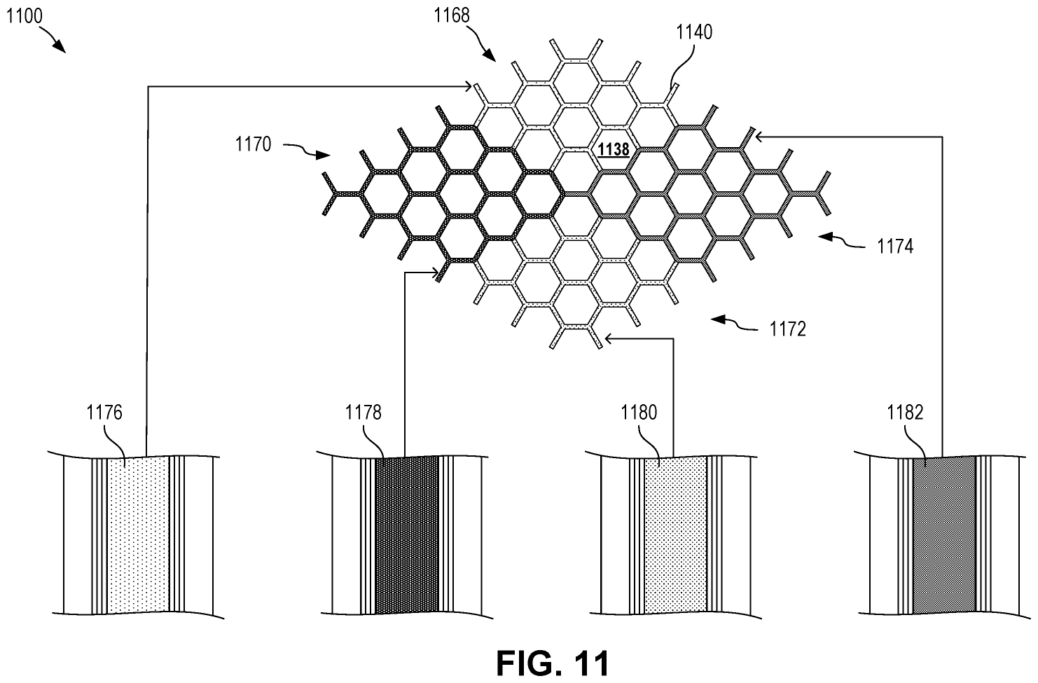

FIG. 11 is a schematic diagram depicting a multiplexed reagent-loaded reaction zone 1100 according to certain aspects of the present disclosure. The reaction zone 1100 can be reaction zone 230 of FIG. 2. The reaction zone 1100 can include passages 1140 defined by pillars 1138. The reaction zone 1100 can be separated into multiple regions, such as a first region 1168, second region 1170, third region 1172, and fourth region 1174. Any number of regions can be used, including two, three, or more than four. Regions can be contiguous with other regions at multiple points, such as depicted in FIG. 11. However, in some cases, regions of the reaction zone 1100 can be fluidly connected at single points, such as in the case of a tree-branching fluid network.

As depicted in FIG. 11, first region 1168 can be pre-loaded with first materials 1176, which may include reagents (e.g., PCR reagents) and/or nucleic acid probes, second region 1170 can be pre-loaded with second materials 1178, third region 1172 can be pre-loaded with third materials 1180, and fourth region 1174 can be pre-loaded with fourth materials 1182. First, second, third, and fourth materials 1176, 1178, 1180, 1182 can include different combinations of reagents and/or nucleic acid probes such that each of the regions 1168, 1170, 1172, 1174 contain unique reagents and/or nucleic acid probes.

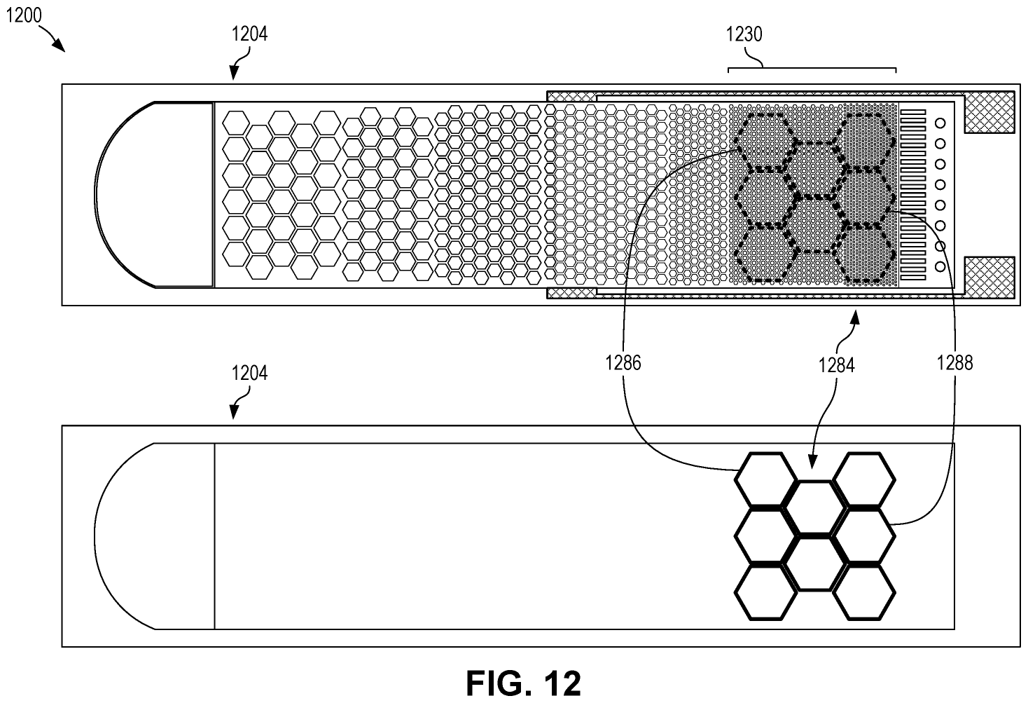

FIG. 12 is set of schematic top view diagrams 1200 depicting a diagnostic chip 1204 with a multiplex reagent-loaded reagent zone according to certain aspects of the present disclosure. The top view of FIG. 12 depicts a schematic diagram of a diagnostic chip 1204, such as diagnostic chip 204 of FIG. 2, with a multiplex array 1284 of multiplexing regions 1286, 1288. As each of the regions 1286, 1288 of the multiplex array 1284 can be preloaded with unique reagents and/or nucleic acid probes, the results of unique assays can be interpreted by independently interpreting the specific regions 1286, 1288 of the chip.

The bottom view of FIG. 12 depicts the same diagnostic chip 1204 as the top view, however with certain elements removed to more clearly depict the multiplex array 1284.

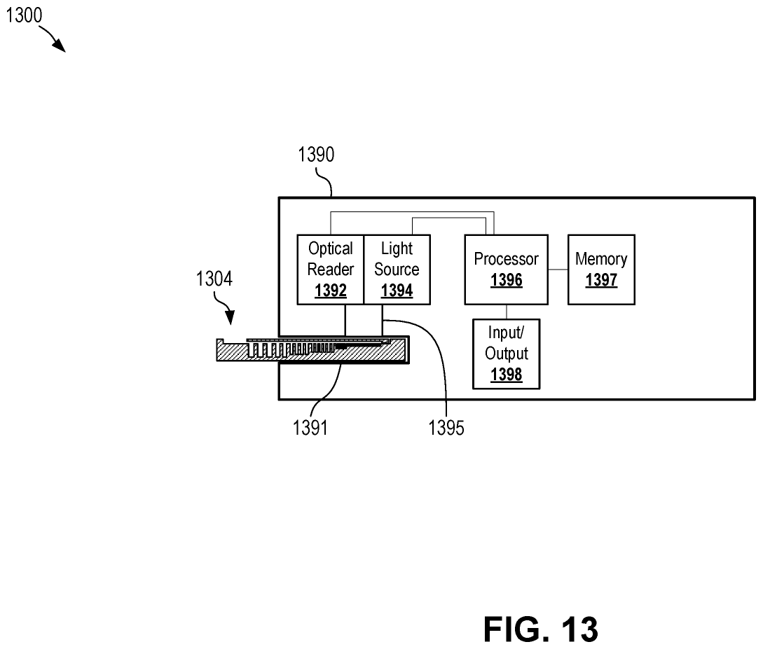

FIG. 13 is a schematic diagram depicting a processing device 1390 for processing diagnostic chips 1304 according to certain aspects of the present disclosure. The processing device 1390 can include a receptacle 1391 for receiving the diagnostic chip 1304. The processing device 1390 can include a processor 1396 coupled to memory 1397 (e.g., for storing data and/or processing instructions). The processor 1396 can be coupled to an input/output device 1398, such as a touchscreen, a camera, a keyboard, or any other suitable input or output device. The processor 1396 can be coupled to a light source 1394 for illuminating the plasmonic materials of the diagnostic chip 1304, such as directly or through a light pipe 1395 or other suitable light directing device. Light from the light source 1394 can induce the generation of surface plasmons in the diagnostic chip 1304, as described herein. The processor 1396 can be coupled to an optical reader 1392 to read the diagnostic chip 1304. The optical reader 1392 can be any suitable device for receiving and/or recording optical energy, such as a camera or other image sensor capable of detecting fluorescence. The optical reader 1392 can be optically coupled to the diagnostic chip 1304 via a light pipe 1395 or other light directing device, although that need not be the case.

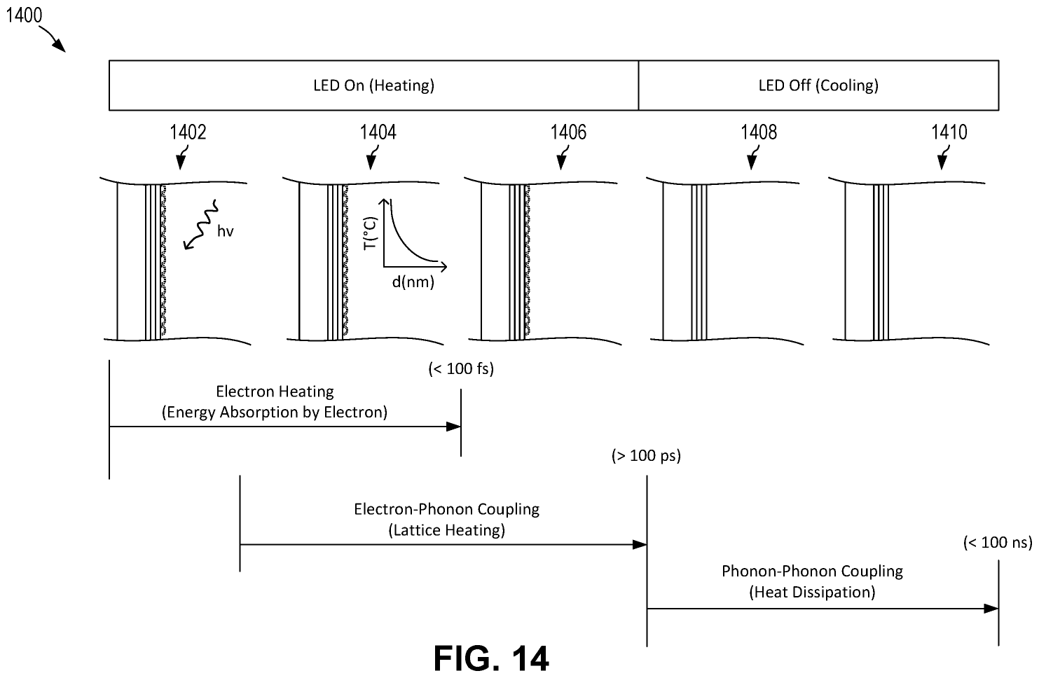

FIG. 14 is a schematic diagram 1400 depicting plasmonic heating and cooling according to certain aspects of the present disclosure. Image 1402 depicts light energy generating surface plasmons in the plasmonic material of a wall of a passage in a fluid network, such as the fluid network 321 of the diagnostic chip 304 of FIG. 3. Surface plasmons induce electron heating, which continues through image 1404 and 1406. In image 1404, surface plasmons have induced substantial heating within the plasmonic material and electron-phonon coupling have started to induce lattice heating. The chart within image 1404 depicts the high temperatures generated near the plasmonic material, which dissipate with an increase in distance (d) from the plasmonic material. At image 1406, the lattice heating has sufficiently heated the region within the plasmonic nanocavity, thus heating the materials within.

At image 1408, the light has ceased and surface plasmon generation has ceased. The pillar begins to cool quickly and dissipate heat from the materials within the region within the plasmonic nanocavity. At image 1410, sufficient heat has been removed from the plasmonic nanocavity to return the plasmonic nanocavity to a desired temperature. In some cases, the process of heating and cooling depicted in diagram 1400 can be repeated several times to facilitate PCR.

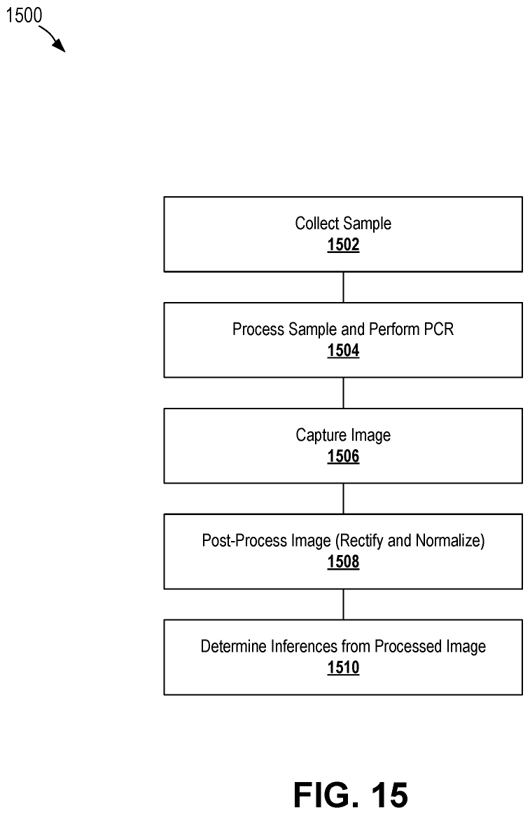

FIG. 15 is a flowchart depicting a process 1500 for collecting and analyzing a sample according to certain aspects of the present disclosure. At block 1502, a sample is collected. The sample can be collected directly within a diagnostic chip, or collected elsewhere and deposited into a diagnostic chip. At block 1504, the sample can be processed and PCR can be performed. As described herein, processing the sample and performing PCR can be performed entirely within a diagnostic chip, with the assistance of light energy to induce surface plasmons to facilitate PCR. In some cases, certain aspects of block 1504 can be facilitated using a processing device, such as the application of light energy and optionally the application of electrical energy (e.g., to facilitate electrochemical lysing).

At block 1506, an image can be captured. The image can be captured of fluorescence in the various plasmonic nanocavities of the diagnostic chip, after PCR has been performed. The image can depict in which plasmonic nanocavities nucleic acids have replicated and/or in which plasmonic nanocavities replicated nucleic acids match the provided nucleic acid probe. Image capture can be performed with any suitable device, such as a camera, a smartphone, or other equipment. However, in some cases, image capture can occur within a processing device that also provides light energy for generation of surface plasmons. In some cases, in addition to capturing an image at block 1506, additional metadata can be collected, such as information about the patient or the sample.

At block 1508, post processing of the raw image data can be performed. Post processing can include performing various image-processing and data-processing actions, such as rectification, normalization, or other actions. In some cases, post-processing can be performed within the processing device, although that need not be the case. Post-processing can be offloaded to another computing device, such as a smartphone or server, for post-processing.

At block 1510, inferences can be determined from the processed image. Inferences can be determined through processing in a trained model. The trained model can be a deep neural network or other suitable machine-learning-trained model. The trained model can be trained on previous PCR data collected from previously processed diagnostic chips. In some cases, inferences can make use of additional metadata collected during or before capturing the image of the diagnostic chip.

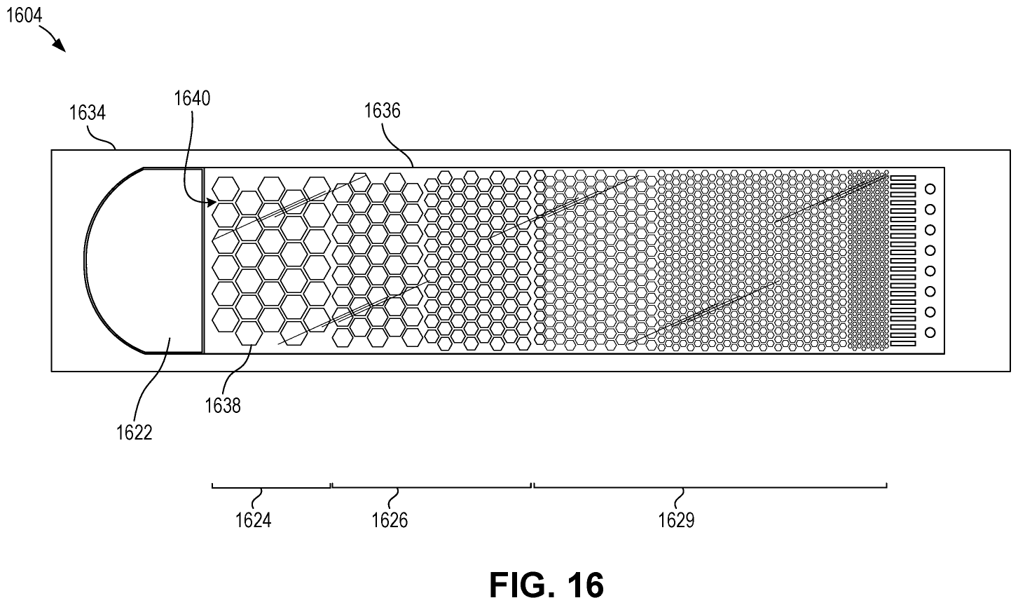

FIG. 16 is a top view of a diagnostic chip 1604 with thermal lysing according to certain aspects of the present disclosure. The diagnostic chip 1604 can be similar to diagnostic chip 204 of FIG. 2, however with a different layout of passages 1640 and without electrodes. The diagnostic chip 1604 can contain a sample input port 1622, a separation zone 1624, a pumping zone 1626, a window 1636, and numerous pillars 1638 defining passages 1640. However, the passages 1640 can be slightly larger in the distal zones (e.g., a combined lysing and reaction zone 1629) to accommodate the larger lysable particles. Lysable particles can fill into the plasmonic nanocavities of the combined lysing and reaction zone 1629. Application of light can induce generation of surface plasmons at the walls of the plasmonic nanocavities, which can increase temperatures in the plasmonic nanocavities and can facilitate lysing of the lysable particles. Since the lysable particles can thus be lysed using thermal and plasmonic energy, there may be no need for electrodes to generate any hydroxide ions.

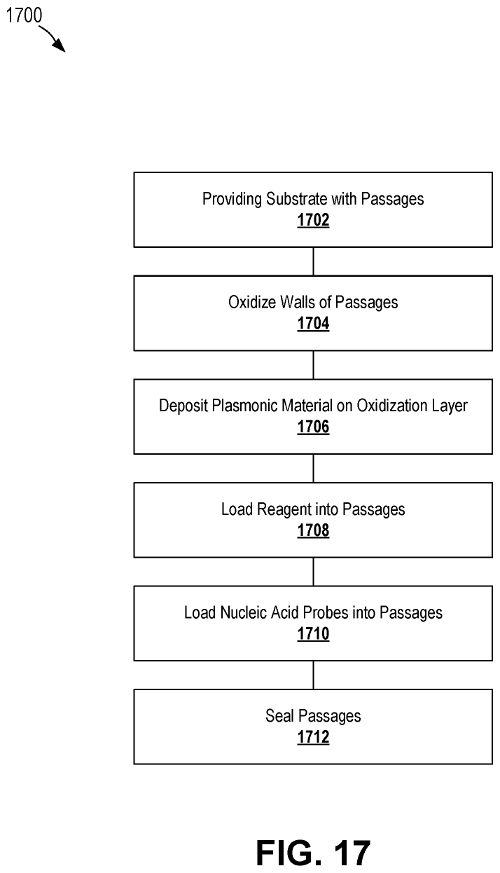

FIG. 17 is a flowchart depicting a process 1700 for preparing a diagnostic chip according to certain aspects of the present disclosure. At block 1702, a substrate with passages is provided. The passages can be cut into the substrate or formed through the building of pillars on a surface of the substrate, or through any suitable technique. The passages can include walls defining the passages.

At block 1704, the walls of the passages can be oxidized. Oxidization of the walls of the passages can produce an oxidization layer. In some cases, the walls of the passageways can be silicon and the oxidization layer can be SiO₂.

At block 1706, the plasmonic material can be deposited. The plasmonic material can be deposited on the oxidization layer, although that need not always be the case, and the plasmonic material may instead be deposited on another layer, such as directly on the walls of the passages. Any suitable plasmonic material can be used, such as gold.

At block 1708, reagent can be loaded into the passages. Reagent can be loaded into the passages in the reaction zone. In some cases, loading reagent can include loading unique reagents in different regions of the reaction zone to form a multiplex array. In some cases, the reagent can be a lyophilized reagent. At block 1710, nucleic acid probes can be loaded into the passages. In some cases, loading nucleic acid probes can include loading unique nucleic acid probes in different regions of the reaction zone to form a multiplex array. In some cases, the nucleic acid probes can be lyophilized nucleic acid probes. In some cases, block 1708 and 1710 can be performed simultaneously, such as in cases where the reagents and nucleic acid probes are pre-mixed.

In some cases, reagents and/or nucleic acid probes can be deposited by directing the materials through the fluid network until they reach the desired locations. However, in some cases, the materials can be deposited directly into the desired plasmonic nanocavities through an opening, such as an opening at the top of each plasmonic nanocavity. In such cases, an optional block 1712 can include sealing the passages of the fluid network, such as by sealing the top of each of the plasmonic nanocavities. In some cases, sealing at block 1712 can include sealing a window onto the diagnostic chip.

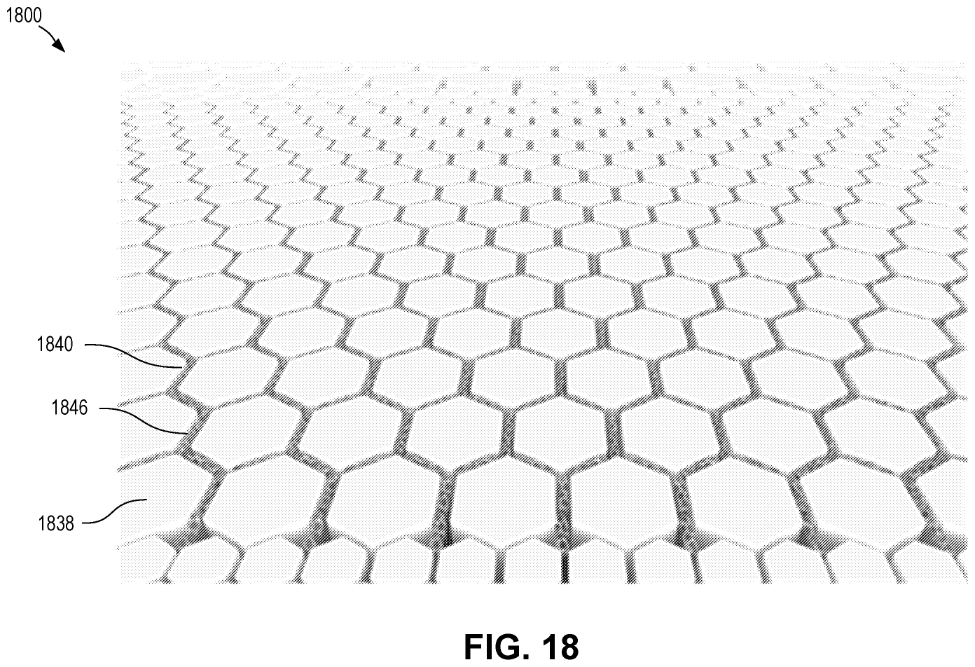

FIG. 18 is a schematic diagram depicting a lysis zone 1800 of a diagnostic chip according to certain aspects of the present disclosure. Pillars 1838 can form passages 1840 of a fluid network. Within the lysis zone 1800, pathogens 1846 can be located within the passages 1840 of the fluid network of the diagnostic chip. Lysis has been induced, such as through chemical, electrochemical, or other techniques, resulting in lysed pathogens 1846.

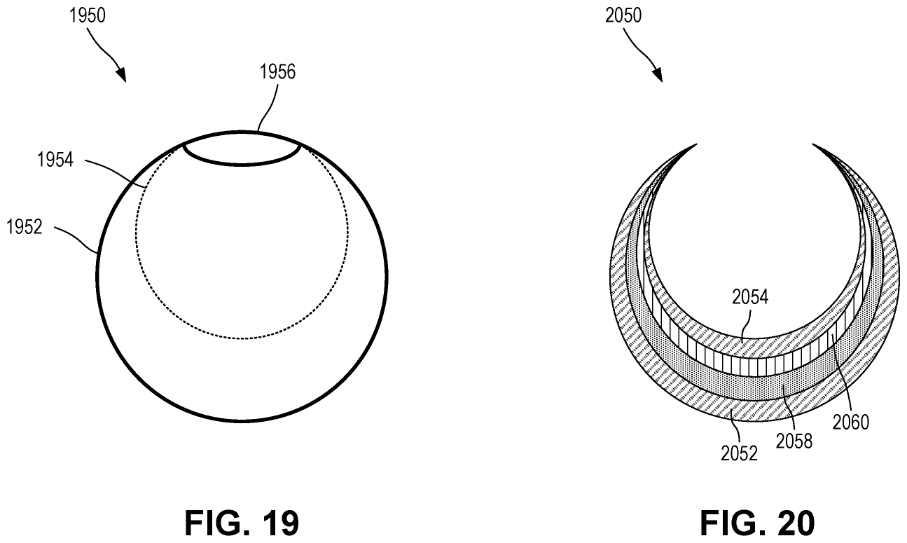

FIG. 19 is a side view of a nanocrescent antenna 1950 according to certain aspects of the present disclosure. The nanocrescent antenna 1950 is a type of nanoantenna. A nanocrescent antenna 1950 can have an outer surface 1952 and an inner surface 1954. The nanocescent antenna 1950, or at least the outer surface 1952 and inner surface 1954, can be made of a plasmonic material, such as gold. The inner surface 1954 can define a cavity within the nanocrescent antenna 1950 exposed to the exterior via opening 1956. The depth of the cavity and/or the diameter of the opening 1956 can be adjusted to tune the nanoantenna. As depicted in FIG. 19, the nanocrescent antenna 1950 is generally spherical in shape. In some cases, nanoantennae can take shapes other than crescent-shaped, such as described elsewhere herein. A nanocrescent antenna 1950 can be especially useful for leveraging QBET principles.

FIG. 20 is a side cutaway view of a multilayer nanocrescent antenna 2050 according to certain aspects of the present disclosure. The multilayer nanocrescent antenna 2050 can be nanocrescent antenna 1950 of FIG. 19. The multilayer nanocescent antenna 2050 can include an outer layer 2052 and inner layer 2054, one or both of which can be formed of a plasmonic material, such as gold. An optional middle plasmonic layer 2058 can be positioned between the outer layer 2052 and inner layer 2054 and can be formed of another plasmonic material, such as silver. An optional middle ferrous layer 2060 can be positioned between the outer layer 2052 and the inner layer 2054 and can be formed of a ferrous material, such as iron. In an example, a multilayer nanocrescent antenna 2050 can be formed of an outer layer 2052 of gold, an middle plasmonic layer 2058 of silver, an middle ferrous layer 2060 of iron, and an inner layer 2054 of gold.

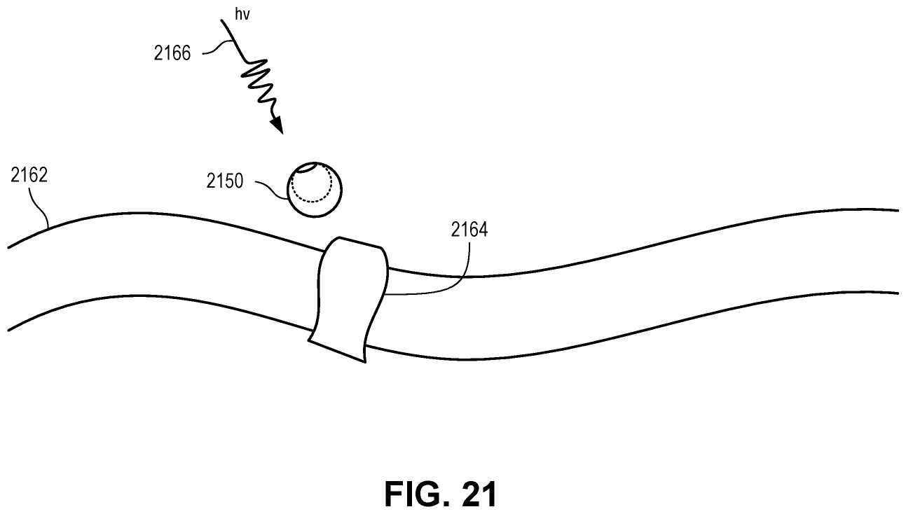

FIG. 21 is a schematic side view of a nanoantenna 2150 usable to effect an ion channel 2164 of a membrane 2162 according to certain aspects of the present disclosure. The nanoantenna 2150 can be a nanocrescent antenna, such as nanocrescent antennae 1950, 2050 of FIG. 19, 20. Membrane 2162 can be any suitable biological membrane, such as a mitochondrial membrane or a membrane of a nerve cell. Ion channel 2164 can be any suitable ion channel of the membrane 2162, such as a cytochrome c protein complex of a mitochondrial membrane or any suitable ion channel in a neural membrane.

By applying suitable electromagnetic energy 2166 (e.g., light) to a nanoantenna 2150 proximate the ion channel 2164, the efficacy and/or operation of the ion channel 2164 can be modulated. Leveraging QBET principles, a nanoantenna 2150 impacted with electromagnetic energy 2166 can provide sufficient coupling with the electron transfer of the ion channel 2164 to adjust the functioning of the ion channel 2164, and thus increase or decrease transfer of ions across the membrane 2162.

In some cases, applying electromagnetic energy 2166 to the nanoantenna 2150 and measuring and rebounding or reflecting energy can be used to sense the conditions proximate the nanoantenna 2150, or more specifically, the operation of the ion channel 2164. Thus, a nanoantenna 2150 can be used as a sensor for the target ion channel 2164.

The foregoing description of the embodiments, including illustrated embodiments, has been presented only for the purpose of illustration and description and is not intended to be exhaustive or limiting to the precise forms disclosed. Numerous modifications, adaptations, and uses thereof will be apparent to those skilled in the art.

As used below, any reference to a series of examples is to be understood as a reference to each of those examples disjunctively (e.g., “Examples 1-4” is to be understood as “Examples 1, 2, 3, or 4”).

Example 1 is an ultrafast diagnostic device, comprising: a sample input for accepting a sample containing desired particles; a fluid network comprising a plurality of fluid pathways extending distally away from the sample input, wherein the fluid network comprises: a separation zone comprising one or more cavities configured to retain undesired particles from the sample, wherein the one or more cavities are coupled to the plurality of fluid pathways to permit passage of the desired particles through the separation zone; a reaction zone comprising a plurality of plasmonic nanocavities fluidly coupled to the plurality of fluid pathways, wherein each plasmonic nanocavity comprises opposing walls each comprising a layer of plasmonic material, wherein the opposing walls of the plasmonic nanocavity are spaced apart by a distance of approximately 5 nanometers or less; and a window permitting transmission of light into and out of the plurality of plasmonic nanocavities of the reaction zone, wherein the window permits transmission of light having wavelengths in the visible spectrum, the infrared spectrum, or the ultraviolet spectrum.

Example 2 is the ultrafast diagnostic device of example 1, wherein the opposing walls of the plasmonic nanocavities are spaced apart by a distance at or less than 3 nm.

Example 3 is the ultrafast diagnostic device of examples 1 or 2, wherein the fluid network further comprises: a pumping zone comprising one or more capillaries sized to induce motive force in the sample through capillary action upon introduction of the sample into the sample input.

Example 4 is the ultrafast diagnostic device of examples 1-3, wherein the one or more cavities of the separation zone form a functional gradient having openings sized to accept the undesired particles.

Example 5 is the ultrafast diagnostic device of example 4, wherein each of the one or more cavities of the separation zone extend from the one of the plurality of fluid pathways within the separation zone to permit gravitational settling of the undesired particles within the cavity.

Example 6 is the ultrafast diagnostic device of examples 1-5, wherein the fluid network further comprises: a lysing zone comprising one or more cavities for receiving lysable particles of the sample and a set of electrodes positioned to supply an electrical current at the one or more cavities to facilitate lysing the lysable particles, wherein the desired particles of the sample are located within the lysable particles.

Example 7 is the ultrafast diagnostic device of example 6, further comprising a set of external electrical contacts operably coupled to the set of electrodes of the lysing zone, wherein the set of external electrical contacts are couplable to an external device for supplying the electrical current to the set of electrodes.

Example 8 is the ultrafast diagnostic device of examples 1-7, wherein the one or more cavities of the separation zone are sized to accept blood cells.

Example 9 is the ultrafast diagnostic device of examples 1-8, wherein each plasmonic nanocavity of the reaction zone is sized to accept a single double helix of nucleic acid.

Example 10 is the ultrafast diagnostic device of examples 1-9, wherein the opposing walls of each plasmonic nanocavity of the reaction zone further comprises a layer of dielectric material.

Example 11 is the ultrafast diagnostic device of examples 1-10, wherein each plasmonic nanocavity of the reaction zone further comprises a polymerase reagent.

Example 12 is the ultrafast diagnostic device of example 11, wherein the polymerase reagent is a lyophilized polymerase reagent.

Example 13 is a method of preparing materials, comprising: receiving a sample containing desired particles at a sample input of a diagnostic device; conveying the desired particles through a fluid network in a distal direction, wherein conveying the desired particles through the fluid network comprises: conveying the sample into a separation zone, wherein conveying the sample into the separation zone comprises separating undesired particles from the sample and conveying the desired particles through the separation zone; and conveying the desired particles into plasmonic nanocavities of a reaction zone, wherein each plasmonic nanocavity comprises opposing walls each comprising a layer of plasmonic material, wherein the opposing walls of each plasmonic nanocavity are spaced apart by a distance of approximately 5 nanometers or less; and transmitting light into each of the plasmonic nanocavities through a window, wherein the light is selected from the group consisting of infrared light, visible light, and ultraviolet light.

Example 14 is the method of example 13, wherein conveying the desired particles into plasmonic nanocavities of the reaction zone further comprises conveying each of the desired particles to a unique one of the plasmonic nanocavities.

Example 15 is the method of example 14, wherein conveying each of the desired particles to unique ones of the plasmonic nanocavities comprises conveying double helixes of nucleic acids to unique ones of the plasmonic nanocavities.

Example 16 is the method of examples 13-15, wherein conveying the desired particles through the fluid network further comprises pumping the desired particles through the fluid network using capillary action.

Example 17 is the method of examples 13-16, wherein conveying the sample into the separation zone further comprises conveying the sample through a functional gradient having openings sized to accept the undesired particles, wherein separating the undesired particles from the sample comprises trapping the undesired particles in the functional gradient.

Example 18 is the method of example 17, wherein trapping the undesired particles in the functional gradient includes permitting the undesired particles to gravitationally settle into one or more cavities of the separation zone.

Example 19 is the method of examples 13-18, further comprising lysing lysable particles of the sample to release the desired particles, wherein lysing lysable particles occurs within a lysing zone of the fluid network located distally from the separation zone.

Example 20 is the method of example 19, wherein lysing the lysable particles comprises applying an electrical current to the separation zone.

Example 21 is the method of examples 13-20, wherein separating undesired particles from the sample comprises separating blood cells from a blood sample.

Example 22 is the method of examples 13-21, wherein the opposing walls of each plasmonic nanocavity of the reaction zone further comprises a layer of dielectric material.

Example 23 is a diagnostic system, comprising: a diagnostic chip comprising a sample input for accepting a sample containing desired particles and a fluid network, the fluid network comprising: a separation zone comprising one or more cavities configured to retain undesired particles from the sample, wherein the one or more cavities are coupled to a plurality of fluid pathways of the fluid network to permit passage of the desired particles through the separation zone; and a reaction zone comprising a plurality of plasmonic nanocavities fluidly coupled to the plurality of fluid pathways, wherein each plasmonic nanocavity comprises opposing walls each comprising a layer of plasmonic material, wherein the opposing walls of the plasmonic nanocavity are spaced apart by a distance of approximately 5 nanometers or less; and a processing device for processing the diagnostic chip, wherein the processing device comprises: a receptacle sized to accept the diagnostic chip; a light source positioned to illuminate the reaction zone when the diagnostic chip is positioned within the receptacle; and a processor coupled to the light source to control application of light to the reaction zone to induce plasmonic resonance in the plasmonic nanocavities of the reaction zone.

Example 24 is the diagnostic system of example 23, wherein the processing device further comprises a detector coupled to the processor and positioned to detect electromagnetic emissions from the reaction zone of the diagnostic chip.

Example 25 is a diagnostic system comprising: a diagnostic chip comprising the ultrafast diagnostic device of any of example(s)s 1-12; and a processing device for processing the diagnostic chip, wherein the processing device comprises: a receptacle sized to accept the diagnostic chip; a light source positioned to illuminate the reaction zone when the diagnostic chip is positioned within the receptacle; and a processor coupled to the light source to control application of light to the reaction zone to induce plasmonic resonance in the plasmonic nanocavities of the reaction zone.

Example 26 is the diagnostic system of example 25, wherein the processing device further comprises a detector coupled to the processor and positioned to detect electromagnetic emissions from the reaction zone of the diagnostic chip.

Example 27 is a diagnostic method, comprising: preparing materials according to the method of any of examples 13-22; and inducing plasmonic resonance in the plasmonic nanocavities, wherein inducing plasmonic resonance comprises illuminating the reaction zone with light.

Example 28 is the diagnostic method of example 27, further comprising: cyclically heating and cooling the desired particles in the reaction zone for a plurality of cycles, wherein heating the desired particles comprises inducing the plasmonic resonance, and wherein cooling the desired particles comprise ceasing illuminating the reaction zone with light.

Example 29 is the diagnostic method of examples 27 or 28, further comprising: detecting electromagnetic emissions from the reaction zone.

Example 30 is the diagnostic method of example 29, wherein illuminating the reaction zone with light includes using a light source, and wherein detecting electromagnetic emissions comprises illuminating the reaction zone using the light source to evoke the electromagnetic emissions.

Example 31 is the diagnostic method of examples 29 or 30, further comprising: storing the electromagnetic emissions as image data; and analyzing the image data to determine a diagnostic inference.

Example 32 is the diagnostic method of example 31, wherein analyzing the image data comprises using a deep neural network to determine the diagnostic inference.

Example 33 is the diagnostic method of examples 31 or 32, wherein analyzing the image data comprises: transmitting the image data using a network interface, wherein transmitting the image data using the network interface results in the image data being applied to a deep neural network to generate the diagnostic inference when the transmitted image data is received; and receiving the diagnostic inference using the network interface.

Example 34 is a method of preparing a chip, comprising: providing a substrate having a plurality of walls defining a plurality of passages, wherein the plurality of passages includes one or more passages having a width of at or less than 100 nm; oxidizing surfaces of the plurality of walls to form an oxidization layer; depositing a plasmonic material on the oxidization layer; and loading reagent into the plurality of passages.

Example 35 is the method of example 34, wherein providing the substrate comprises providing a silicon substrate, and wherein oxidizing the surfaces of the plurality of walls forms a layer of silicon dioxide.

Example 36 is the method of examples 34 or 35, wherein the plurality of passages includes one or more passages having a width of at or less than 40 nm.

Example 37 is the method of examples 34 or 35, wherein the plurality of passages includes one or more passages having a width of at or less than 10 nm.

Example 38 is the method of examples 34-37, wherein depositing the plasmonic material comprises depositing gold.

Example 39 is the method of examples 34-38, wherein loading reagent comprises loading lyophilized reagent into the plurality of passages.

Example 40 is the method of example 39, wherein loading lyophilized reagent comprises loading lyophilized polymerase chain reaction reagents.

Example 41 is the method of examples 34-40, wherein loading reagent comprises: loading a first reagent into a first set of the plurality of passages; and loading a second reagent into a second set of the plurality of passages.

Example 42 is the method of examples 34-41, further comprising loading nucleic acid probes into the plurality of passages.

Example 43 is the method of example 42, wherein loading nucleic acid probes comprises: loading a first nucleic acid probe into a first set of the plurality of passages; and loading a second nucleic acid probe into a second set of the plurality of passages.

Example 44 is the method of examples 34-43, wherein each of the plurality of passages have an open top, and wherein the method further comprises sealing the open top of each of the plurality of passages.

Example 45 is the method of example 44, wherein sealing the open top of each of the plurality of passages comprises sealing each of the plurality of passages with a window permitting transmission of light into and out of the passage.

Example 46 is a method for imaging electron transfer, comprising: positioning a plasmonic nanoantenna adjacent target tissue; irradiating the plasmonic nanoantenna with electromagnetic energy to induce the plasmonic nanoantenna to emit emitted electromagnetic energy, wherein the emitted electromagnetic energy is associated with electron transfer of the target tissue; measuring emitted electromagnetic energy from the plasmonic nanoantenna.

Example 47 is the method of example(s) 46, wherein the target tissue is an ion channel of a membrane.

Example 48 is the method of example(s) 47, wherein the ion channel is a cytocrome c protein of a mitochondrial membrane.

Example 49 is the method of example(s) 46-48, wherein irradiating the plasmonic nanoantenna with electromagnetic energy comprises irradiating the plasmonic nanoantenna with light.

Example 50 is a method for biological intervention, comprising: positioning a plasmonic nanoantenna adjacent target tissue; and manipulating electron transfer of the target tissue by irradiating the plasmonic nanoantenna with electromagnetic energy.

Example 51 is the method of example(s) 50, wherein the target tissue is an ion channel of a membrane.

Example 52 is the method of example(s) 51, wherein the ion channel is a cytocrome c protein of a mitochondrial membrane.

Example 53 is the method of example(s) 50-52, wherein irradiating the plasmonic nanoantenna with electromagnetic energy comprises irradiating the plasmonic nanoantenna with light.

What is claimed is:

1. An ultrafast diagnostic device, comprising:
a sample input for accepting a sample containing desired particles;
a fluid network comprising a plurality of fluid pathways extending distally away from the sample input, wherein the fluid network comprises:
a separation zone comprising one or more cavities configured to retain undesired particles from the sample, wherein the one or more cavities are coupled to the plurality of fluid pathways to permit passage of the desired particles through the separation zone;
a reaction zone comprising a plurality of plasmonic nanocavities fluidly coupled to the plurality of fluid pathways, wherein each plasmonic nanocavity comprises opposing walls each comprising a layer of plasmonic material, wherein the opposing walls of the plasmonic nanocavity are spaced apart by a distance of approximately 5 nanometers or less; and
a window permitting transmission of light into and out of the plurality of plasmonic nanocavities of the reaction zone, wherein the window permits transmission of light having wavelengths in the visible spectrum, the infrared spectrum, or the ultraviolet spectrum.

2. The ultrafast diagnostic device of claim 1, wherein the opposing walls of the plasmonic nanocavities are spaced apart by a distance at or less than 3 nm.

3. The ultrafast diagnostic device of claim 1, wherein the fluid network further comprises:
a pumping zone comprising one or more capillaries sized to induce motive force in the sample through capillary action upon introduction of the sample into the sample input.

4. The ultrafast diagnostic device of claim 1, wherein the one or more cavities of the separation zone form a functional gradient having openings sized to accept the undesired particles.

5. The ultrafast diagnostic device of claim 4, wherein each of the one or more cavities of the separation zone extend from the one of the plurality of fluid pathways within the separation zone to permit gravitational settling of the undesired particles within the cavity.

6. The ultrafast diagnostic device of claim 1, wherein the fluid network further comprises:
a lysing zone comprising one or more cavities for receiving lysable particles of the sample and a set of electrodes positioned to supply an electrical current at the one or more cavities to facilitate lysing the lysable particles, wherein the desired particles of the sample are located within the lysable particles.

7. The ultrafast diagnostic device of claim 6, further comprising a set of external electrical contacts operably coupled to the set of electrodes of the lysing zone, wherein the set of external electrical contacts are couplable to an external device for supplying the electrical current to the set of electrodes.

8. The ultrafast diagnostic device of claim 1, wherein the one or more cavities of the separation zone are sized to accept blood cells.

9. The ultrafast diagnostic device of claim 1, wherein each plasmonic nanocavity of the reaction zone is sized to accept a single double helix of nucleic acid.

10. The ultrafast diagnostic device of claim 1, wherein the opposing walls of each plasmonic nanocavity of the reaction zone further comprises a layer of dielectric material.

11. The ultrafast diagnostic device of claim 1, wherein each plasmonic nanocavity of the reaction zone further comprises a polymerase reagent.

12. The ultrafast diagnostic device of claim 11, wherein the polymerase reagent is a lyophilized polymerase reagent.

13. A method of preparing materials, comprising:
receiving a sample containing desired particles at a sample input of a diagnostic device;
conveying the desired particles through a fluid network in a distal direction, wherein conveying the desired particles through the fluid network comprises:
conveying the sample into a separation zone, wherein conveying the sample into the separation zone comprises separating undesired particles from the sample and conveying the desired particles through the separation zone; and
conveying the desired particles into plasmonic nanocavities of a reaction zone, wherein each plasmonic nanocavity comprises opposing walls each comprising a layer of plasmonic material, wherein the opposing walls of each plasmonic nanocavity are spaced apart by a distance of approximately 5 nanometers or less; and
transmitting light into each of the plasmonic nanocavities through a window, wherein the light is selected from the group consisting of infrared light, visible light, and ultraviolet light.

14. The method of claim 13, wherein conveying the desired particles into plasmonic nanocavities of the reaction zone further comprises conveying each of the desired particles to a unique one of the plasmonic nanocavities.

15. The method of claim 14, wherein conveying each of the desired particles to unique ones of the plasmonic nanocavities comprises conveying double helixes of nucleic acids to unique ones of the plasmonic nanocavities.

16. The method of claim 13, wherein conveying the desired particles through the fluid network further comprises pumping the desired particles through the fluid network using capillary action.

17. The method of claim 13, wherein conveying the sample into the separation zone further comprises conveying the sample through a functional gradient having openings sized to accept the undesired particles, wherein separating the undesired particles from the sample comprises trapping the undesired particles in the functional gradient.

18. The method of claim 17, wherein trapping the undesired particles in the functional gradient includes permitting the undesired particles to gravitationally settle into one or more cavities of the separation zone.

19. The method of claim 13, further comprising lysing lysable particles of the sample to release the desired particles, wherein lysing lysable particles occurs within a lysing zone of the fluid network located distally from the separation zone.

20. The method of claim 19, wherein lysing the lysable particles comprises applying an electrical current to the separation zone.

21. The method of claim 13, wherein separating undesired particles from the sample comprises separating blood cells from a blood sample.

22. The method of claim 13, wherein the opposing walls of each plasmonic nanocavity of the reaction zone further comprises a layer of dielectric material.

23. A diagnostic system, comprising:
a diagnostic chip comprising a sample input for accepting a sample containing desired particles and a fluid network, the fluid network comprising:
a separation zone comprising one or more cavities configured to retain undesired particles from the sample, wherein the one or more cavities are coupled to a plurality of fluid pathways of the fluid network to permit passage of the desired particles through the separation zone; and
a reaction zone comprising a plurality of plasmonic nanocavities fluidly coupled to the plurality of fluid pathways, wherein each plasmonic nanocavity comprises opposing walls each comprising a layer of plasmonic material, wherein the opposing walls of the plasmonic nanocavity are spaced apart by a distance of approximately 5 nanometers or less; and
a processing device for processing the diagnostic chip, wherein the processing device comprises:
a receptacle sized to accept the diagnostic chip;
a light source positioned to illuminate the reaction zone when the diagnostic chip is positioned within the receptacle; and
a processor coupled to the light source to control application of light to the reaction zone to induce plasmonic resonance in the plasmonic nanocavities of the reaction zone.

24. The diagnostic system of claim 23, wherein the processing device further comprises a detector coupled to the processor and positioned to detect electromagnetic emissions from the reaction zone of the diagnostic chip.

25. A diagnostic system comprising:
a diagnostic chip comprising the ultrafast diagnostic device of any of claims 1-12; and
a processing device for processing the diagnostic chip, wherein the processing device comprises:
a receptacle sized to accept the diagnostic chip;
a light source positioned to illuminate the reaction zone when the diagnostic chip is positioned within the receptacle; and
a processor coupled to the light source to control application of light to the reaction zone to induce plasmonic resonance in the plasmonic nanocavities of the reaction zone.

26. The diagnostic system of claim 25, wherein the processing device further comprises a detector coupled to the processor and positioned to detect electromagnetic emissions from the reaction zone of the diagnostic chip.

27. A diagnostic method, comprising:
preparing materials according to the method of any of claims 13-22; and
inducing plasmonic resonance in the plasmonic nanocavities, wherein inducing plasmonic resonance comprises illuminating the reaction zone with light.

28. The diagnostic method of claim 27, further comprising:
cyclically heating and cooling the desired particles in the reaction zone for a plurality of cycles, wherein heating the desired particles comprises inducing the plasmonic resonance, and wherein cooling the desired particles comprise ceasing illuminating the reaction zone with light.

29. The diagnostic method of claim 27, further comprising:
detecting electromagnetic emissions from the reaction zone.

30. The diagnostic method of claim 29, wherein illuminating the reaction zone with light includes using a light source, and wherein detecting electromagnetic emissions comprises illuminating the reaction zone using the light source to evoke the electromagnetic emissions.

31. The diagnostic method of claim 29, further comprising:
storing the electromagnetic emissions as image data; and
analyzing the image data to determine a diagnostic inference.

32. The diagnostic method of claim 31, wherein analyzing the image data comprises using a deep neural network to determine the diagnostic inference.

33. The diagnostic method of claim 31, wherein analyzing the image data comprises:
transmitting the image data using a network interface, wherein transmitting the image data using the network interface results in the image data being applied to a deep neural network to generate the diagnostic inference when the transmitted image data is received; and
receiving the diagnostic inference using the network interface.

34. A method of preparing a chip, comprising:
providing a substrate having a plurality of walls defining a plurality of passages, wherein the plurality of passages includes one or more passages having a width of at or less than 100 nm;
oxidizing surfaces of the plurality of walls to form an oxidization layer;
depositing a plasmonic material on the oxidization layer; and
loading reagent into the plurality of passages.

35. The method of claim 34, wherein providing the substrate comprises providing a silicon substrate, and wherein oxidizing the surfaces of the plurality of walls forms a layer of silicon dioxide.

36. The method of claim 34, wherein the plurality of passages includes one or more passages having a width of at or less than 40 nm.

37. The method of claim 34, wherein the plurality of passages includes one or more passages having a width of at or less than 10 nm.

38. The method of claim 34, wherein depositing the plasmonic material comprises depositing gold.

39. The method of claim 34, wherein loading reagent comprises loading lyophilized reagent into the plurality of passages.

40. The method of claim 39, wherein loading lyophilized reagent comprises loading lyophilized polymerase chain reaction reagents.

41. The method of claim 34, wherein loading reagent comprises:
loading a first reagent into a first set of the plurality of passages; and
loading a second reagent into a second set of the plurality of passages.

42. The method of claim 34, further comprising loading nucleic acid probes into the plurality of passages.

43. The method of claim 42, wherein loading nucleic acid probes comprises:
loading a first nucleic acid probe into a first set of the plurality of passages; and
loading a second nucleic acid probe into a second set of the plurality of passages.

44. The method of claim 34, wherein each of the plurality of passages have an open top, and wherein the method further comprises sealing the open top of each of the plurality of passages.

45. The method of claim 44, wherein sealing the open top of each of the plurality of passages comprises sealing each of the plurality of passages with a window permitting transmission of light into and out of the passage.

46. A method for imaging electron transfer, comprising:
positioning a plasmonic nanoantenna adjacent target tissue;
irradiating the plasmonic nanoantenna with electromagnetic energy to induce the plasmonic nanoantenna to emit emitted electromagnetic energy, wherein the emitted electromagnetic energy is associated with electron transfer of the target tissue;
measuring emitted electromagnetic energy from the plasmonic nanoantenna.

47. The method of claim 46, wherein the target tissue is an ion channel of a membrane.

48. The method of claim 47, wherein the ion channel is a cytocrome c protein of a mitochondrial membrane.

49. The method of claim 46, wherein irradiating the plasmonic nanoantenna with electromagnetic energy comprises irradiating the plasmonic nanoantenna with light.

50. A method for biological intervention, comprising:
positioning a plasmonic nanoantenna adjacent target tissue; and
manipulating electron transfer of the target tissue by irradiating the plasmonic nanoantenna with electromagnetic energy.

51. The method of claim 50, wherein the target tissue is an ion channel of a membrane.

52. The method of claim 51, wherein the ion channel is a cytocrome c protein of a mitochondrial membrane.

53. The method of claim 50, wherein irradiating the plasmonic nanoantenna with electromagnetic energy comprises irradiating the plasmonic nanoantenna with light.
